(19) United States
(12) Patent Application Publication
(10) Pub. No.: US 2006/0057104 A1
(43) Pub. Date: Mar. 16, 2006

(54) Methods for stimulating tlr irf3 pathways for inducing anti-microbial, anti-inflammatory and anticancer responses

(75) Inventors: Genhong Cheng, Calabasas, CA (US); Robert L. Modlin, Sherman Oaks, CA (US); Sagar Vaidya, Yucca Valley, CA (US); Sean Doyle, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE Regents of the University of California Office of the President, Oakland, CA (US)

(21) Appl. No.: 10/512,124

(22) PCT Filed: Apr. 24, 2003
(86) PCT No.: PCT/US03/12751

§ 371 Date: Aug. 26, 2005

Related U.S. Application Data
(60) Provisional application No. 60/375,489, filed on Apr. 24, 2002.

(51) Int. Cl. A61K 48/00; A61K 38/21; A61K 39/00; A61K 31/739; A61K 31/4745; A61K 31/337

(52) U.S. Cl. 424/85.4; 514/54; 514/44; 424/185.1; 514/291; 514/449

(57) ABSTRACT

The present invention provides methods for stimulating Toll-like receptors (TLR) to activate IRF (e.g., an IRF3) and signaling pathway, and thereby directing an antimicrobial activity. The present invention also provides methods for identifying agents that bind and/or stimulate TLR and mediate induction of an IRF3 pathway, thereby directing an antimicrobial activity. Additionally, the invention provides methods and agents for suppressing stimulation of TLR, thereby directing an anti-inflammatory response.

This application claims priority to provisional application, U.S. Ser. No. 60/375,489 filed Apr. 24, 2002, the contents of which are hereby incorporated by reference in their entirety into this application.

Throughout this application various publications are referenced. The disclosures of these publications are hereby incorporated by reference in their entirety into this application in order to more fully describe the state of the art to which the invention pertains.

### FIELD OF INVENTION

The present invention relates to methods for stimulating Toll-like receptors to activate IRF (e.g., an IRF3) and signaling pathway, and directing an antimicrobial activity.

### BACKGROUND OF THE INVENTION

Challenge by invading pathogens has led multicellular organisms to develop a number of defensive measures for the recognition and clearance of infectious agents. The innate immune system is capable of recognizing a wide variety of pathogens and rapidly induces a number of antimicrobial and inflammatory responses. Toll-like receptors (TLR) play a critical role in innate immunity by recognizing structurally conserved bacterial and viral components termed pathogen-associated molecular patterns (PAMPs) (Medzhitov, R., and Janeway, C. J. (1998), Semin. Immunol. 10, 351-353). Ten TLRs have been cloned in mammals, and each receptor appears to be involved in the recognition of a unique set of PAMPs. While the focus of many studies has been mainly on bacterial components, TLR3, TLR4, and TLR7 have been shown to mediate the response to the viral-associated PAMPs: the double-stranded RNA analog poly I:C; the F protein of Respiratory Syncytial Virus (RSV); and the antiviral therapeutic compounds, the imidazoquinolines, respectively (Alexopoulou, L., Holt, A. C., Medzhitov, R., and Flavell, R. A. (2001), Nature 413, 732-738; Hemmi, H., Kaisho, T., Takeuchi, O., Sato, S., Sanjo, H., Hoshino, K., Horiuchi, T., Tomizawa, H., Takeda, K., and Akira, S. (2002), Nat. Immunol. 3, 196-200; Kopp, E. B., and Medzhitov, R. (1999), Curr. Opin. Immunol. 11, 13-18; Kurt-Jones, E., Popova, L., Kwinn, L., Haynes, L., Jones, L., Tripp, R., Walsh, E., Freeman, M., Golenbock, D., Anderson, L., and Finberg, R. (2000), Nat. Immunol. 1, 398-401; Takeuchi, O., and Akira, S. (2001), Int. Immunopharmacology 1, 625-635; Takeuchi, O., Hoshino, K., Kawai, T., Sanjo, H., Takada, H., Ogawa, T., Takeda, K., and Akira, S. (1999), Immunity 11, 443-451).

TLRs activate signaling through the Toll/IL-1R (TIR) domain found in the cytoplasmic tails of these proteins (Akira, S. (2000), Biochem. Soc. Trans. 28, 551-556; Akira, S., Takeda, K., and Kaisho, T. (2001), Nat. Immunol. 2, 675-680; Guha, M., and Mackman, N. (2001), Cell. Signal. 13, 85-94; Takeuchi, O., and Akira, S. (2001), Int. Immunopharmacology 1, 625-635). Receptor activation triggers binding of the adaptor protein MyD88 (myeloid differentiation factor 88) to the TIR domain, allowing for interaction and autophosphorylation of IRAK (IL-1R-associated kinase) and subsequent activation of tumor necrosis factor receptor-associated factor 6 (TRAF6), leading to the activation of the NF-κB, JNK, PI3K, p38, and ERK pathways (Takeuchi, O., and Akira, S. (2001), Int. Immunopharmacology 1, 625-635; Ardeshna, K. M., Pizzey, A. R., Devereux, S., and Khwaja, A. (2000), Blood 96, 1039-1046).

While all TLRs originally appeared to activate the same signaling pathways to initiate the inflammatory response, recent studies have indicated that the functional roles of TLR3 and TLR4 are more complex for several reasons. First, TLR4 has been shown to mediate the response to a wide variety of ligands other than lipopolyssaccharide (LPS), including Gram-positive lipoteichoic acids, the cancer chemotherapeutic Taxol, and the F protein of RSV (Kurt-Jones, E., Popova, L., Kwinn, L., Haynes, L., Jones, L., Tripp, R., Walsh, E., Freeman, M., Golenbock, D., Anderson, L., and Finberg, R. (2000), Nat. Immunol. 1, 398-401; Medzhitov, R., and Janeway, C. J. (1998), Semin. Immunol. 10, 351-353; Takeuchi, O., and Akira, S. (2001), Int. Immunopharmacology 1, 625-635; Takeuchi, O., Hoshino, K., Kawai, T., Sanjo, H., Takada, H., Ogawa, T., Takeda, K., and Akira, S. (1999), Immunity 11, 443-451). More perplexing is the fact that TLR4−/− mice have been shown to have increased susceptibility to infection by RSV, while no such finding has yet been reported in models of bacterial infection (Haynes, L. M., Moore, D. D., Kurt-Jones, E. A., Finberg, R. W., Anderreceptorson, L. J., and Tripp, R. A. (2001), J. Virol. 75, 10730-10737). Also, TLR3 and TLR4 have been shown to activate NF-κB in cells lacking MyD88, albeit with delayed kinetics (Alexopoulou, L., Holt, A. C., Medzhitov, R., and Flavell, R. A. (2001), Nature 413, 732-738; Horng, T., Barton, G. M., and Medzhitov, R. (2001), Nat. Immunol. 2, 835-841; Kawai, T., Adachi, O., Ogawa, T., Takeda, K., and Akira, S. (1999), Immunity 11, 115-122). Recent reports indicate that the newly cloned TIR domain-containing molecule TIRAP/Mal may function as a second adaptor for TLR3 and TLR4 and direct activation of downstream signaling molecules in the absence of MyD88 (Fitzgerald, K. A., Pallson-McDermott, E. M., Bowie, A. G., Jeffries, C. A., Mansell, A. S., Brady, G., Brint, E., Dunne, A., Gray, P., and Harte, M. T. (2001), Nature 413, 78-83; Homg, T., Barton, G. M., and Medzhitov, R. (2001), Nat. Immunol. 2, 835-841).

Importantly, recent reports have described a role for interferon regulatory factor 3 (IRF3) in the TLR4 signaling cascade (Kawai, T., Takeuchi, O., Fujita, T., Inoue, J.-I., Mühlradt, P. F., Sato, S., Hoshino, K., and Akira, S. (2001), J. Immunol. 167, 5887-5894; Navarro, L., and David, M. (1999), J. Biol. Chem. 274, 35535-35538). IRF3 is an important transcriptional regulator of the anti-viral immune response. Through an unknown mechanism, viral infection causes IRF3 to become phosphorylated and migrate to the nucleus where it participates in the activation of a complex positive feedback loop between Type I IFNs and IRF family members, leading to induction of an antigrowth, antiviral response (Sato, M., Taniguchi, T., and Tanaka, N. (2001), Cytokine Growth Factor Rev. 12, 133-142; Taniguchi, T., Ogasawara, K., Takaoka, A., and Tanaka, N. (2001), Annu. Rev. Immunol. 19, 623-655; Taniguchi, T., and Takaoka, A. (2002), Curr. Opin. Immunol. 14, 111-116). TLR4-mediated nuclear translocation of IRF3 has been shown to occur in a MyD88-independent fashion and to induce binding to interferon-stimulated response elements (ISRE) in vitro at 2 hr poststimulation (Kawai, T., Takeuchi, O., Fujita, T., Inoue, J.-I., Muhlradt, P. F., Sato, S., Hoshino, K., and Akira, S. (2001), J. Immunol. 167, 5887-5894). However, the functional role of IRF3 in TLR3- or TLR4-induced gene expression remains largely undetermined.

Interestingly, members of the tumor necrosis factor receptor (TNFR) family use pathways similar to those utilized by TLRs to mediate quite different biological effects. One TNFR, CD40, has been shown to be intimately involved in the adaptive immune response (Foy et al., 1996). At the molecular level, we have found that CD40 stimulation activates the NF-κB, JNK, p38, and PI3K pathways, while, functionally, CD40 is required for germinal center formation and affinity maturation (Gordon, J., and Pound, J. D. (2000), Immunology 100, 269-280). Currently, the molecular mechanisms that differentiate TLR-mediated innate and TNFR-mediated adaptive immune responses are unknown.

The present invention provides methods of stimulating TLRs, and thereby activating IRF3 and NF-κB pathways. The inventive methods are useful for inducing an immune response against antigens of interest which are associated with, e.g., microbial or viral infections, and antigens associated with inflammatory responses and cancers.

### SUMMARY OF THE INVENTION

The present invention provides methods for stimulating TLRs, and thereby activating IRF (e.g., IRF3) and NF-κB pathways. In one embodiment, a molecule that binds and/or stimulates TLR is a TLR ligand. The TLR ligand of the invention includes but is not limited to bacterial antigens, LPS, lipid A, taxol, viral antigens, RSV F protein, double stranded RNA, poly I:C, or small molecules.

The invention further provides a method for activating IRF3 in a cell comprising contacting the cell with a molecule that binds and or stimulates a TLR, thereby activating IRF3 in the cell.

The methods of the invention induce nuclear translocation of an IRF (e.g.,IRF3) and NF-κB which leads to the upregulation of a set of primary response genes. The primary response genes of the inventive method include but are not limited to IFIT1, ISG15, RANTES, IP10, and IFNβ.

The present invention provides methods for increasing the activity of a cellular protein which mediates a primary anti-viral response, where the cellular protein includes IP10, RANTES, IFNβ, ISG15, and IFIT1.

In one embodiment, IFNβ activates STAT1, and induces expression of secondary response genes. The secondary response genes of the invention include but are not limited to Mx1, IFI1, IFI204, or IRF7.

The present invention also provides agents that bind and/or stimulate TLR and mediate induction of IRF e.g., an IRF3 pathway. These agents include but are not limited to nucleic acids, such as double stranded RNA, poly I:C, proteins, viral antigens such as F protein of RSV (RSV F protein), bacterial antigens, such as lipopolysacchrides (LPS), Gram-positive lipoteichoic acid, lipid A, cancer chemotherapeutic taxol, or small molecules, such as the imidazoquinoline like compounds.

The present invention further provides methods for screening and identifying agents that bind and/or stimulate TLR and mediate induction of an IRF (e.g., IRF3) pathway.

The invention further provides agents that directly or indirectly bind and/or suppress TLR stimulation, thereby inhibiting intracellular signaling pathway, i.e., induction of IRF e.g., an IRF3 pathway. The agents that suppress TLR stimulation include but are not limited to soluble TLR (e.g., soluble TLR3 and TLR4), anti-TLR antibodies, anti-IFN (e.g., anti-IFNβ) antibodies, anti-LPS antibodies and anti-PAMP antibodies. Additionally, small molecules that inhibit stimulation of TLR may be used to suppress stimulation of TLR.

The present invention further provides methods for screening and identifying agents that bind and/or inhibit TLR and thereby inhibit an IRF (e.g., IRF3) pathway.

The inventive methods are useful for inducing an immune response against antigens of interest which are associated with, e.g., microbial or viral infections, and antigens associated with inflammatory responses and cancers.

The present invention further provides pharmaceutical compositions comprising the compositions that bind and/or stimulate TLR and mediate induction of IRF (e.g., IRF3) pathway. Additionally, the invention provides pharmaceutical compositions comprising the compositions that bind and/or inhibit stimulation of TLR, and inhibit activation of IRF (e.g., IRF3) pathways.

### BRIEF DESCRIPTIONS OF THE FIGURES

FIG. 1: LPS but not CD40L upregulates a set of genes previously characterized as “interferon-responsive,” as described in Example 1, infra.

(A) Primary murine B cells were stimulated with LPS (20 μg/mL) or a soluble CD8/CD40L chimera (300 ng/mL) and RNA was collected at indicated time points and used to conduct microarray analysis. Genes previous characterized as “interferon-responsive” were further subdivided and absolute expression changes were displayed using the Treeview Program. Expression changes: Red—Induced, Green—Repressed, Black—No Change. Selected time points were hybridized to microarrays in duplicate to ensure reproducibility.

(B) Line charts display temporal expression pattern of selected genes from (A).

FIG. 2: Characterization of TLR3/TLR4-Primary Response Genes, as described in Example 1, infra.

(A) RAW 264.7 macrophages were stimulated with LPS (100 ng/mL) for the indicated time points, RNA was harvested, and then analyzed by Northern blotting (left) using a cDNA probe for RANTES. Twelve micrograms of total RNA was loaded in each lane. CHX indicates 30 min. pre-treatment and costimulation with cycloheximide (20 μg/mL). Radioactive signal from each lane was quantified (right) using STORM™ and ImageQuant software (Molecular Dynamics).

(B) Primary murine bone marrow-derived macrophages (BMM's) were stimulated with LPS (100 ng/mL) for the indicated time points, RNA was harvested, and then analyzed by quantitative real-time PCR (Q-PCR) for RANTES expression (left) and 18S RNA (right) expression. Experiments were conducted in triplicate and standard deviation expressed as error bars. All Q-PCR data in this report represented as relative expression units unless otherwise indicated.

(C) BMM's were stimulated with the following TLR-agonists: lipid A (1 ng/mL), peptidoglycan (PGN) (10 μg/mL), poly I:C (I μg/ml), or CpG (100 nM) for 30 min, and cell extracts were used for an in vitro kinase assay using GST-c-jun as a substrate (upper left), identical simulations were repeated for I hr, and RNA was harvested, and used for Q-PCR analysis.

(D) Summary of TLR3/TLR4-primary response genes (see text for details). Primary-Reponse defined as upregulated by LPS (100 ng/mL) at 2 hr in the presence of cycloheximide (20 μg/mL)

^{Φ}Schematic representation of gene promoters created by using Celera web-based genomic database to obtain 5′ regulatory region, followed by theoretical analysis using TESS promoter analysis software (http://www.cbil.upenn.edu/tess/) and the TRANSFAC transcription factor database. Relevant consensus sequence matches are in accordance with published literature.

^{δ}Induced synergistically by LPS and CHX; DC, dendritic cells; PBL, peripheral blood leukocytes

FIG. 3: IRF3 and NF-κB are involved in TLR3/TLR4-mediated gene activation, as described in Example 1, infra.

(A) BMM's were treated for indicated time points with lipid A (1 ng/mL), peptidoglycan (PGN) (20 μg/mL), poly I:C (10 μg/mL), or CpG (100 nM). Cells were fractionated and 30 μg of nuclear extract was analyzed by SDS-PAGE immunoblotting for IRF3 nuclear translocation followed by stripping and reprobing with p65 and USF2.

(B) Purity of cellular fractionation was tested by probing identical blots for the nuclear protein, USF2, or the cytoplasmic protein tubulin.

(C) CAT reporter assay showing LPS-induced transactivation of the IP10 promoter. RAW 264.7 macrophages were transiently transfected with 1 μg of −243-IP10-pCAT and co-transfected with 6 μg of pCDNA3 (mock), pEBB-IRF3-DBD or pCDNA3-IκBm-ER (IκB-DA) as labeled. Six hours posttransfection, cells were treated with media or LPS (100 ng/mL) for 24 hr and 30 μg of protein was used for each CAT reaction. Results are representative of three separate experiments.

FIG. 4: NF-κB is required for activation of Primary Response Genes, while IRF3 mediates TLR3/TLR4 specificity, as described in Example 1, infra.

(A) RAW 264.7 clones stably expressing pCDNA3 (mock) or pCDNA3-IκBm-ER (IκB-DA) were treated for 30 min with LPS (100 ng/ml), tamoxifen (200 nM), or both, and NF-κB activity was assayed by EMSA.

(B) RAW-mock and RAW-IκB-DA cell lines were pretreated with tamoxifen (200 nM) for 2 hr and were stimulated with LPS (100 ng/ml) for the indicated time points. RNA was harvested and used for Q-PCR analysis.

(C) Stable expression of pEBB-IRF3 or pEBB-IRF3-DBD in RAW 264.7 cells was detected by Western blotting.

(D) RAW 264.7 macrophages expressing the IRF3 constructs in (C) were stimulated with LPS (100 ng/ml) or poly I:C (10 μg/ml), and RNA was harvested and used for Q-PCR analysis.

(E) RAW wild-type or IRF3-expressing macrophages were stimulated with PGN (20 μg/ml), and RNA was harvested and used for Q-PCR analysis.

FIG. 5: Characterization of TLR3/TLR4 Secondary Response Genes. BMMs were stimulated with LPS (100 ng/ml) for the indicated time points, and RNA was harvested and then analyzed by Q-PCR, as described in Example 1, infra.

(A) Mx1 gene induction expressed in relative expression units.

(B) Kinetics of activation of IFNβ versus secondary response genes expressed as fold change (note: log scale).

(C) Summary of TLR3/TLR4 secondary response genes (see text for details). Secondary-reponse defined as upregulated by LPS (100 ng/ml) at 4 hr but blocked in the presence of cycloheximide (20 μg/ml). Confers resistance to bacterial and viral infection were based on studies using transgenic mice. N/D, not determined; HIN, Hematopoietic Interferon-inducible Nuclear Protein.

FIG. 6: TLR3/TLR4 stimulation induces production of IFNβ and activates antiviral responses, as described in Example 1, infra.

(A) BMMs were stimulated for the indicated time points with the following TLR-agonists: lipid A (1 ng/ml), PGN (20 μg/ml), poly I:C (1 μg/ml), or CpG (100 nM), and RNA was harvested and used for Q-PCR analysis.

(B) BMMs were treated for indicated time points with the following TLR-agonists: lipid A (1 ng/mI), PGN (50 μg/ml), or CpG (100 nM), and 20 μg of protein extract was analyzed by SDS-PAGE immunoblotting using antibody specific for phosphorylated-STAT1 (Y701) or total STAT1.

(C) BMMs were treated for 30 min with cell-free conditioned media (CM) from BMMs treated for 2 hr with media or 100 ng/ml LPS in the presence or absence of 20 μg/ml blocking antibodies or nonspecific rabbit IgG (RIgG) as indicated. RNA was harvested and used for Q-PCR analysis.

(D) STAT1 activation is blocked by anti-IFNβ. BMMs were treated for 30 min with CM as described in (C), and STAT1 phosphorylation was then assayed as in (B).

(E) BMMs were infected with MHV68 (moi=5) and simultaneously treated with the indicated TLR ligands (100 nM CpG; 10, 1, or 0.1 ng/ml lipid A; 10 μg/ml PGN; or 1, 0.1, or 0.01 μg/ml poly I:C). After 48 hr, cells were harvested and analyzed for MHV68 replication proteins by immunoblotting using rabbit anti-MHV68 antibodies.

(F) NIH 3T3 cells were pretreated for 3 hr with conditioned media from BMM treated with LPS (100 ng/ml), lipid A (1 ng/ml), PGN (10 μg/ml), or poly I:C (1 μg/ml) in the presence or absence of anti-IFNα/β or nonspecific rabbit IgG (20 μg/ml). Cells were then infected with MHV68 (moi=1) for 24 hr. MHV68 replication was assayed as described in (E). All results are representative of at least three separate experiments.

FIG. 7: Model of TLR3/TLR4-Specific Antiviral Gene Program. Activation of TLR3 and TLR4 by poly I:C and LPS, respectively, induces the nuclear translocation of IRF3 and NF-κB, which leads to the upregulation of a set of primary response genes. IFNβ is one important cytokine that is produced, activates STAT1, and induces expression of genes that can inhibit viral replication in uninfected cells, as described in Example 1, infra.

FIG. 8: TLR3 is a more potent inducer of antiviral gene expression than TLR4. Murine BMMs were stimulated with poly(I:C) (10 μg/ml) or lipid A (1 ng/ml) for the indicated times. Total RiNA was isolated and converted to cDNA for quantitative real-time PCR analysis using primers specific for IFN-β, IFI-204, IP10, IκBα, or L32. Experiments were repeated three times, and the data are presented in relative expression units on a log scale, as described in Example 2, infra.

FIG. 9: MyD88, but not TIRAP/MAL, directly interacts with TLR3. 293T lysate-containing MyD88 or flag-TIRAP/MAL was incubated with the intracellular domains of TLR3 and TLR4 fused to GST and immobilized on glutathione beads. TLR-MyD88 interaction was determined by Western blotting using a polyclonal anti-MyD88 Ab (upper panel). TLR-TIRAP/MAL interaction was determined by Western blotting using an anti-flag Ab to detect flag-TIRAP/MAL (middle panel). Equal amounts of beads containing GST-TLR3 or -TLR4 intracellular domains were boiled and the eluted proteins were size-fractionated by SDS-PAGE. Coomassie blue staining (lower panel) was used to ensure that comparable amounts of GST-TLR protein were loaded on the beads. The data represent three independent experiments, as described in Example 2, infra.

FIG. 10: The TIRAP/MAL inhibitory peptide is able to block TLR4 but not TLR3 transactivation of IFN-β and IL-6, as well as IFN-β-mediated activation of STAT1. BMMs were pretreated with the TIRAP/MAL peptide (20 μM) or DMSO for 1 h and then stimulated with lipid A (1 ng/ml), poly I:C (1 μg/ml), or medium alone for 2 h, as described in Example 2, infra.

(A) IFN-β, IL-6, and L32 mRNA levels were assayed by quantitative real-time PCR. All samples were run in duplicate or triplicate, and data are presented in relative expression units.

(B) STAT1 activation was determined by Western blotting analysis to detect phosphorylated STAT1. For STAT1 experiments, lipid A was used at 10 ng/ml, and poly I:C was administered at 100 and 10 ng/ml. Total STAT1 was also assayed to ensure equal loading. The data are representative of three independent experiments.

FIG. 11: Both TLR3 and TLR4 ligands can induce expression of TLR3 through IFN-β. Primary macrophage cells derived from bone marrow cells were stimulated with poly I:C (1 μg/ml) or lipid A (1 ng/ml) for the indicated times, as described in Example 2, infra.

(A) Quantitative real-time PCR was used to assay the expression levels of TLR3, TLR4, MyD88, and TIRAP/MAL.

(B) TLR3 and TLR4 mRNA levels were also assessed in cells deficient in IFNAR, and cells stimulated with rIFN-β (10, 100, and 1000 U). Experiments were repeated at least two separate times, and data are presented in relative expression units. L32 was used to normalize all samples.

FIG. 12: TLR3 and TLR4 induce both IFN-β-enhanced and IFN-β-dependent antiviral genes.

Both wild-type cells and cells deficient in IFNAR were stimulated with poly I:C (1 μg/ml) or lipid A (1 ng/ml) for either 1 or 4 h. IFN-β, IP10, IFI-204, ICAM1, and L32 mRNA levels were assessed. Data are representative of three independent experiments and presented in relative expression units, as described in Example 2, infra.

FIG. 13: Both TLR3 and TLR4 fail to activate STAT1 and induce the antiviral gene program in IFNAR−/− primary macrophage cells. BMMs from both wild-type and IFNAR−/− mice were stimulated with lipid A (1 ng/ml), poly I:C (1 μg/ml), CpG (100 nM), or fresh medium (M) for the indicated time periods, as described in Example 2, infra.

(A): Cell lysates were subjected to western blotting analyses to detect phosphorylated STAT1 (P-STAT1) and total STAT1.

(B) For viral replication assays, BMMs were simultaneously stimulated with PAMPs (10, 1, or 0.1 ng/ml lipid A, or 1, 0.1, or 0.01 μg/ml poly(I:C)) and infected with MHV68 using a multiplicity of infection of five. Cell lysates were harvested at 48 h postinfection and subjected to Western blotting analysis using an Ab specific to the MHV68 protein M9. Actin levels were also assayed to ensure equal loading. The data represent two independent experiments.

FIG. 14: TLR3/4 activation leads to an IFN-dependent G1/S block in murine macrophage cells, as shown in Example 3, infra.

FIG. 15: TLR3/4 specificity upregulate genes involved in G1/S transition, as shown in Example 3, infra.

FIG. 16: TLR3 activation decreases apoptosis in the RAW 264.7 macrophage cell line as shown in Example 4, infra.

FIG. 17: Infection with live Listeria monocytogenes (LM) activates the IRF3-IFNβ pathway and may influence development of adaptive immune responses, as shown in Example 5, infra.

### DETAILED DESCRIPTION OF THE INVENTION

### DEFINITIONS

All scientific and technical terms used in this application have meanings commonly used in the art unless otherwise specified. As used in this application, the following words or phrases have the meanings specified.

As used herein, the term “TLR” refers to Toll-like receptors which play a critical role in innate immunity by recognizing structurally conserved pathogen-associated molecular patterns (PAMPs). Examples of TLR include TLR1, TLR2, TLR3, TLR4, TLR5, TLR6, TLR7, TLR8, TLR9, and TLR10.

As used herein, the term “PAMPs” refers to highly conserved structural motifs expressed by microbial pathogens. PAMPs include various bacterial cell wall components such as lipopolysaccharides (LPS), peptidoglycans and lipopeptides, as well as flagellin, bacterial DNA and viral double-stranded RNA.

As used herein, the term “primary response genes” refers to nucleotide gene sequences encoding primary response proteins. The expression of primary response genes does not require new protein synthesis.

As used herein, the term “secondary response genes” refers to nucleotide gene sequences encoding secondary response proteins. The expression of secondary response genes requires new protein synthesis.

As used herein, the term “agonist” refers to a molecule that can bind to cellular receptors/proteins (e.g., TLR) and/or directly or indirectly activate intracellular signaling pathways/gene expression.

As used herein, the term “antagonist” refers to a molecule that can bind to cellular receptors/proteins and/or directly or indirectly inhibit intracellular signaling pathways/gene expression.

As used herein, the term “antimicrobial” refers to an agent that inhibits replication or proliferation of a microbial organism, such as bacteria, virus, or fungi.

As used herein, the term “stimulation of TLR” refers to addition of a unique pathogen-associated molecular patterns (PAMPs) that requires a specific Toll-like receptor (TLR) for recognition.

As used herein, the term “inhibition of TLR stimulation” or “suppression of TLR stimulation” refers to addition of molecules that block interaction of TLR with PAMPs.

As used herein, the term “activation” refers to cellular changes in response to an environmental stimulus, resulting in activity of a multitude of biochemical signaling pathways and significant changes in gene expression.

In order that the invention herein described can be more fully understood, the following description is set forth.

### METHODS OF THE INVENTION

The present invention provides methods for stimulating TLR pathways involving activation of IRF3. The methods of the invention comprise contacting a cell that expresses a TLR with a molecule or agent that stimulates a TLR under suitable conditions so that the cell so contacted activates IRF3. The methods for stimulating TLR pathways are useful for inducing an immune response against antigens of interest which are associated with, e.g., microbial infection, such as bacterial or viral infections.

In one embodiment, an agent that binds and/or stimulates TLR is a TLR ligand. The TLR ligand of the invention includes but is not limited to: bacterial antigens, such as lipopolysacchrides (LPS), including Gram-positive lipoteichoic acid, and lipid A; cancer chemotherapeutic agents, such as taxol; viral antigens, such as F protein of RSV (RSV F protein); nucleic acid molecules, such as double stranded RNA, double stranded RNA analogues, poly I:C. Additionally, the agents that bind and/or stimulate TLR include small molecules, including but not limited to imidazoquinoline like compounds may be used.

The methods of the invention induce nuclear translocation of IRF3 and/or NF-κB which leads to the upregulation of a set of primary response genes (as determined by resistance to cycloheximide treatment). The primary response genes of the inventive method include but are not limited to IFIT1, ISG15, RANTES, IP10, and IFNβ.

The secreted IFNβ instigates an autocrine/paracrine loop, activating STAT1, and thereby induces expression of secondary response genes. The secondary response genes of the invention include but are not limited to Mx1, IFI1, IFI204, or IRF7. The expression of secondary response genes can inhibit microbial infection, such as viral or bacterial replication in uninfected cells.

The present invention also provides methods to suppress the TLR stimulation, thereby inhibiting activation of IRF3 pathway. The methods of suppressing TLR stimulation comprise contacting a cell that expresses a TLR with a molecule or agent that suppresses stimulation of TLR under suitable conditions, so that the cell so contacted inhibits activation of IRF3. The methods to suppress TLR stimulation are useful in inducing an anti-inflammatory response.

The inventive methods are useful for inducing an immune response against antigens of interest which are associated with, e.g., microbial infection, such as bacterial or viral infections, and antigens associated with inflammatory responses and cancers.

### COMPOSITIONS OF THE INVENTION

In its various aspects, as described in detail below, the present invention provides agents that bind and/or stimulate TLR. In one embodiment the agent that binds and/or stimulates TLR is a TLR ligand. In specific embodiments the agent of the invention binds and/or stimulates TLR3 or TLR4. The agents include: proteins, peptides, antibodies, nucleic acid molecules, recombinant DNA molecules, small molecules (organic or inorganic compounds). The present invention also includes methods for obtaining and using the compositions of the invention, including screening and diagnostic assays, therapeutic methods, and immunological and nucleic acid-based pharmaceutical or therapeutic assays. In specific embodiments, the TLR ligands include but not limited to bacterial antigens, such as LPS, lipid A, cancer chemotherapeutic agents, such as taxol, viral antigens, such as RSV F protein, double stranded RNA, poly I:C, or small molecules, such as imidazoquinolines.

The present invention also provides molecules or agents that suppress stimulation of TLR stimulation, thereby inhibiting activation of IRF3 pathway.

The molecules or agents that suppress stimulation of TLR include but are not limited to a soluble TLR. As used herein “soluble TLR” means non-cell-surface-bound TLR (e.g., TLR3, TLR4). The soluble TLR may include the extracellular domain of a TLR (e.g., TLR3, TLR4). The extracellular domain of a TLR may be fused to a non-TLR sequence, such as an immunoglobulin (Ig) moiety rendering the fusion molecule soluble, or fragments and derivatives thereof.

An anti-TLR antibody may be used to suppress stimulation of TLR. The TLR antibodies include but are not limited to an anti-TLR3 antibody (IMGENEX, Catalog No. IMG-315) and an anti-TLR4 antibody (IMGENEX, Catalog No. IMG417).

An anti-interferon antibody such as an anti-IFN-β antibody (Buhlmann Diagnostics, Catalog No. EK-IFNB) may also be used to suppress stimulation of TLR.

Further, molecules that block an endotoxin shock, such as an anti-LPS antibody (CalTag, clone 100, Clone MC6; Novus Biologicals, Clone 26-5) may be used to suppress stimulation of TLR. Additionally, molecules that block interaction of TLR with PAMP, such as an anti-PAMP antibody, may be used to suppress stimulation of TLR.

Methods for Inducing an Antiviral, Antimicrobial, or Antifungal Immune Response

The present invention provides methods for stimulating a TLR pathway, comprising: contacting a cell with a TLR ligand of the invention, under suitable conditions so that TLR mediates activation of the IRF3 pathway. The activated IRF3 pathway can activate an IFNβ-dependent anti-viral response pathway.

The present invention provides methods for inducing an antiviral, antimicrobial, or antifungal immune response, comprising: contacting a cell with a TLR ligand of the invention, under suitable conditions so that TLR mediates activation of the IRF3 pathway. In one embodiment, the TLR mediates phosphorylation of the IRF3 protein which activates the IRF3 pathway.

The present invention provides methods for inducing an antiviral, antimicrobial, or antifungal immune response, comprising: contacting a cell with a TLR ligand of the invention, under suitable conditions so that TLR mediates activation of the IRF3 pathway which induces translocation of NF-κB to the nucleus of a cell.

The present invention also provides methods for inducing an antiviral, antimicrobial, or antifungal immune response, comprising: contacting a cell with a TLR ligand of the invention, under suitable conditions so that TLR mediates activation of the IRF3 pathway which increases or upregulates the activity of a cellular protein which mediates a primary anti-viral response. In one embodiment, the cellular protein (e.g., primary protein) includes IP10, RANTES, IFNβ, ISG15, and IFIT1. In another embodiment, the upregulation of the activity of the primary protein includes: increasing the level of the primary protein; increasing the activity of the primary protein (e.g., via phosphorylation); increasing the stability of the primary protein; or decreasing the level of degradation or decreasing the rate of degradation of the primary protein.

The present invention also provides methods for inducing an antiviral, antimicrobial, or antifungal immune response, comprising: contacting a cell with a TLR ligand of the invention, under suitable conditions so that TLR mediates activation of the IRF3 pathway which upregulates the activity of IFNβ.

The present invention also provides methods for inducing an antiviral, antimicrobial, or antifungal immune response, comprising: contacting a cell with a TLR ligand of the invention, under suitable conditions so that TLR mediates activation of the IRF3 pathway which activates a STAT1 protein.

The present invention also provides methods for inducing an antiviral, antimicrobial, or antifungal immune response, comprising: contacting a cell with a TLR ligand of the invention, under suitable conditions so that TLR mediates activation of the IRF3 pathway which upregulates the activity of a secondary anti-viral response protein. In one embodiment the secondary response genes include but are not limited to Mx1, IFI1, IFI204, or IRF7.

In another embodiment, the upregulation of the activity of the secondary anti-viral response protein includes: increasing the RNA transcript level encoding the secondary protein; increasing the transcription of the RNA encoding the secondary protein; increasing the stability of the RNA transcript encoding the secondary protein; or decreasing the level of degradation or decreasing the rate of degradation of the RNA transcript encoding the secondary protein.

In yet another embodiment, the upregulation of the activity of the secondary anti-viral response protein includes: increasing the level of the secondary protein; increasing the activity of the secondary protein (e.g., via phosphorylation); increasing the stability of the secondary protein; or decreasing the level of degradation or decreasing the rate of degradation of the secondary protein.

The present invention also provides methods for inducing an antiviral, antimicrobial, or antifungal immune response, comprising: contacting a cell with a TLR ligand of the invention, under suitable conditions so that the cell so contacted stimulates the TLR, thereby activating an IRF3 pathway and mediating transactivation of primary response genes of the invention, including upregulation of the level of the IFNβ transcript. The transcativation of primary response genes and upregulation of the level of the IFNβ transcript leads to induction of an immune response and expression of secondary response genes, thereby, inhibiting replication of virus, bacteria or fungi in the cell.

Methods for Inducing Anti-Inflammatory Response

The present invention also provides methods for inducing anti-inflammatory responses, comprising: contacting a cell that expresses a TLR with an agent that suppresses stimulation of TLR, under suitable conditions so that the cell so contacted suppresses stimulation of TLR, thereby inhibiting activation of IRF3 pathway.

Inhibition of IRF3 pathway suppresses primary response genes of the invention, including down-regulation of the level of the IFNβ transcript. The suppression of primary response genes and down-regulation of the level of the IFNβ transcript leads to suppression of an immune response, thereby, induction of an anti-inflammatory response in the cell.

Methods for Inhibiting Tumor Growth

The present invention provides methods for inhibiting the growth of a tumor cell, comprising: contacting a cell that expresses a TLR with a TLR ligand of the invention, under suitable conditions so that the cell so contacted stimulates the TLR, thereby activating an IRF3 pathway and mediating transactivation of primary response genes. The transcativation of primary response genes leads to induction of an immune response and expression of secondary response genes, thereby inhibiting the growth of the tumor cell expressing the antigen of interest. An inhibition of tumor growth is assayed by measuring the size and/or volume of the test tumor in a subject administered the molecule of the invention, and comparing the size and/or volume of the test tumor with the size and/or volume of a control tumor. The control tumor is from a different subject which is not administered the molecule of the invention. The growth of the test tumor is inhibited by administration of the molecule of the invention, when there is a measurable difference in size, volume, or growth rate between the test tumor and control tumor.

Screening for TLR Ligands

We provide herein the discovery that TLR3 activates an interferon beta (IFNβ)-dependent anti-viral gene program. This discovery suggests that TLR3 may be a suitable target for the treatment of a variety of viral infections. The natural ligand for TLR3 is the viral product, double-stranded RNA. However, it has been postulated that double-stranded RNA may bind to other cellular receptors leading to unknown biological outcomes.

The present invention provides methods for identifying small molecule agonists of TLR3 that activate only the TLR3 receptor and specifically activate antiviral responses. It has previously been shown that TLR7 binds to members of the antiviral imidazoquinone family of small molecules (imiquimod and R-848) (Hemmi, H. et al., Nature Immunol. (2002) 3 (6): 499). TLR7 is closely related to TLR9, which binds to bacterial DNA motifs. Imidazoquinones have some structural similarities to purine moieties supporting this relationship. TLR3 also binds to nucleotide structures and may also be activated by molecules related to the TLR7 agonists. Accordingly, the present invention provides methods for screening small molecules which can activate the antiviral gene program. In one embodiment, the methods include screening agents that are structurally related to imidazoquinone and/or agents that are structurally unrelated to imidazoquinones.

The screening methods of the invention include providing a combinatorial library containing a large number of compounds (candidate modulator compounds) (Borman, S, C. & E. News, 1999, 70(10), 33-48). Such combinatorial chemical libraries can be screened in one or more assays to identify library members (particular chemical species or subclasses) that exhibit the ability to activate the antiviral gene program (Borman, S., supra; Dagani, R. C & E. News, 1999, 70(10), 51-60). The compounds, so identified, can serve as lead-compounds or can themselves be. used as potential or actual therapeutics.

A combinatorial chemical library is a collection of diverse chemical compounds generated by using either chemical synthesis or biological synthesis, to combine a number of chemical building blocks, such as reagents. For example, a linear combinatorial chemical library, such as a polypeptide library, is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. The methods for preparing a library of complex compounds reminiscent of natural products are described in U.S. Pat. No. 6,448,443. Such combinatorial chemical libraries include, but are not limited to, peptide libraries (see, e.g., U.S. Pat. No. 5,010,175, Furka, Int. J. Pept. Prot. Res., 1991, 37:487-493 and Houghton, et al., Nature, 1991, 354, 84-88). Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 1993, 90, 6909-6913); vinylogous polypeptides (Hagihara, et al., J. Amer. Chem. Soc. 1992, 114, 6568); nonpeptidal peptidomimetics with beta-D-glucose scaffolding (Hirschmann, et al., J. Amer. Chem. Soc., 1992, 114, 9217-9218); analogous organic syntheses of small compound libraries (Chen, et al., J. Amer. Chem. Soc., 1994, 116, 2661; Armstrong, et al. Acc. Chem. Res., 1996, 29, 123-131); or small organic molecule libraries (see, e.g., benzodiazepines, Baum C&E News, 1993, Jan. 18, page 33,); oligocarbamates (Cho, et al., Science, 1993, 261, 1303); and/or peptidyl phosphonates (Campbell, et al., J. Org. Chem. 1994, 59, 658); nucleic acid libraries (see, Seliger, H et al., Nucleosides & Nucleotides, 1997, 16, 703-710); peptide nucleic acid libraries (see, e.g., U.S. Pat. No. 5,539,083); antibody libraries (see, e.g., Vaughn, et al., Nature Biotechnology, 1996, 14(3), 309-314 and PCT/US96/10287); carbohydrate libraries (see, e.g., Liang, et al., Science, 1996, 274, 1520-1522 and U.S. Pat. No. 5,593,853, Nilsson, U J, et al., Combinatorial Chemistry & High Throughput Screening, 1999 2, 335-352; Schweizer, F; Hindsgaul, O. Current Opinion In Chemical Biology, 1999 3, 291-298); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and other similar art.

Methods and apparatus for screening large numbers of chemical compounds using various fluorescent assays, including laser linescan confocal microscope are described in e.g., U.S. Pat. No. 6,400,487. These methods may be used to screen live cell assays. Additionally, Rapid screening methods for activities and selectivities of catalyst libraries using mass spectrometer analysis may be combined with resonance enhanced multiphoton ionization detection methods (U.S. Pat. No. 6,426,226). A multiwell plate scanner for continuous scanning using fluorescent detection methods as described in U.S Pat. No. 6,448,089 can also be used. Further combinatorial libraries of small molecules using fluorescence-activated cell sorting (FACS) technology (U.S. Pat. No. 6,455,263) may be prepared and used for screening assays.

A number of systems for rapidly identifying ligands/small molecules in liquid samples are known (e.g., U.S. Pat. No. 6,472,218), and may be used. Additionally, combinatorial libraries of compounds which are tagged and attached to solid support may be prepared and screened for rapid and non-destructive identification of chemical compounds attached to solid supports (U.S. Pat. No. 6,541,203). Additionally, methods for generating combinatorial libraries of immobilized compounds and screening for biological activity are described in U.S. Pat. No. 6,541,276, and other similar art.

A number of devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

In the high throughput methods of the invention, several thousand different candidate compounds can be screened in a relatively short period of time. For example, each well of a microtiter plate can be used to run a separate assay against a selected potential modulator, or if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day; assay screens for up to about 6,000-20,000, and even up to about 100,000-1,000,000 different candidate modulator compounds are possible using the methods of the invention.

In another embodiment the present invention provides methods for screening extracts derived from therapeutic Chinese herbs in order to test their ability to activate the antiviral program. In yet another embodiment, the methods include screening agents or compounds for bioactivity by assaying for upregulation of the IFNβ transcript in primary bone marrow-derived macrophages. These methods can be used to identify novel antiviral compounds that may be used to help combat diseases such as severe acute respiratory syndrome (SARS) that currently have few therapeutic options.

Multiwell Plates and Arrays

Screening methods can be performed using multiwell plates which are used for many different types of applications, including library generation and storage. Multiwell plates may also be used for gene amplification using the polymerase chain reaction as described in U.S. Pat. No. 5,545,528 entitled Rapid Screening Method of Gene Amplification Products in Polypropylene Plates. Fluorescence based applications for multiwell plates such as these would be suitable with the present inventions.

The screening methods of the present invention can be performed using high throughput or miniaturized formats. Also contemplated are methods using higher density sample processing systems, for example using chips that contain miniaturized microfluidic devices are being developed.

In another aspect of the present invention, many different assays can be employed with the devices and methods disclosed herein, such as biochemical and cell based assays. Fluorescent probes can be substrates for enzymes, dyes, fluorescent proteins and any other moiety that can produce a fluorescent signal under the appropriate conditions.

Fluorescence Measurements

Different types of fluorescent monitoring systems can be used to practice the invention with fluorescent probes, e.g., fluorescent dyes or substrates. Systems dedicated to high throughput screening, e.g., 96-well or greater microtiter plates, may be used. Assays on fluorescent materials are well known in the art (Lakowicz, J. R., Principles of Fluorescence Spectroscopy, New York: Plenum Press (1983).

Fluorescence resonance energy transfer (FRET) may be used as a way of monitoring probes in a sample (cellular or biochemical).

Additionally, ratiometric fluorescent probe system may be used with the invention (e.g., PCT publication WO96/30540). These methods permit gene expression analysis, as it allows sensitive detection and isolation of both expressing and non-expressing single living cells.

Methods for Detecting the Presence of an Analyte in a Sample

The methods of the present invention can be used to detect the presence of an agent that modulates (e.g, inhibits or stimulates) the activity of a target, in a sample. Typically, a target can be a protein such as a cell surface protein, extracellular enzyme or intracellular enzyme. The target protein can be cell-membrane bound, residing in a cell, or free protein extracted from a cell or tissue. A biological process or a target can be assayed in either biochemical assays (targets free of cells), or cell based assays (targets associated with a cell). In one embodiment, the detecting methods comprise contacting a cell expressing a TLR with a candidate agent that may stimulate (modulates) the TLR (target) to activate the IRF3 pathway thereby inducing a secondary anti-viral response, inducing expression of a secondary anti-viral response protein (e.g., Mx1, IFI1, IFI204, or IRF7), or inducing an anti-viral or anti-bacterial, or anti-inflammatory response.

Pharmaceutical Compositions and Kits

The present invention provides pharmaceutical compositions comprising the nucleic acid, protein, lipids, lipopolysaccharides, or small molecules of the invention and agents, identified using the screening methods described herein, in pharmaceutical composition comprising a pharmaceutically acceptable carrier prepared for storage and subsequent administration. The pharmaceutical compositions preferably include suitable carriers, adjuvant, or diluents which include any material which when combined with a molecule of the invention retains the molecule's activity and is non-reactive with the subject's immune system. Acceptable carriers or diluents for therapeutic use are well known in the pharmaceutical art, and are described, for example, in Remington's Pharmaceutical Sciences, Mack Publishing Co. (A. R. Gennaro edit. 1985). Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

The compositions of the present invention may be formulated and used as tablets, capsules or elixirs for oral administration; suppositories for rectal administration; sterile solutions, suspensions for injectable administration; and the like. Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. If desired, absorption enhancing preparations (e.g., liposomes), may be utilized.

The pharmaceutically effective amount of the composition required as a dose will depend on the route of administration, the type of animal being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the invention, the products or compositions can be used alone or in combination with one another, or in combination with other therapeutic or diagnostic agents. These products can be utilized in vivo, ordinarily in a mammal, preferably in a human, or in vitro. In employing them in vivo, the products or compositions can be administered to the mammal in a variety of ways, including parenterally, intravenously, subcutaneously, intramuscularly, colonically, rectally, nasally or intraperitoneally, employing a variety of dosage forms. Such methods may also be applied to testing chemical activity in vivo.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage for the products of the present invention can range broadly depending upon the desired affects and the therapeutic indication.

The exact formulation, route of administration and dosage can be chosen by the individual physician in view of the patient's condition. (See e.g., Fingl et al., in The Pharmacological Basis of Therapeutics, 1975). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th Ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the invention may be formulated in aqueous solutions, preferably in physiologically compatible buffers such as Hanks' solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping, or lyophilizing processes. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

The preferred form depends upon the mode of administration and the therapeutic application. The most effective mode of administration and dosage regimen for the compositions of this invention depends upon the severity and course of the infection or disease, the patient's health and response to treatment and the judgment of the treating physician. Accordingly, the dosages of the compositions should be titrated to the individual patient.

Further provided are kits comprising compositions of the invention, in free form or in pharmaceutically acceptable form. The kit can comprise instructions for its administration. The kits of the invention can be used in any method of the present invention.

Administering to a Subject

The present invention provides methods for administering the compositions of the invention to a subject. The compositions can be administered to the subject by standard routes, such as intravenous (i.v.), intraperitoneal (i.p.), intramuscular (i.m.), subcutaneous, intradermally, and also oral administration, administration by injection, as a suppository, or the implantation of a slow-release device such as a miniosmotic pump. Administration can be performed daily as a single dose, multiple doses, or in continuous dose form. Administration can be at a tumor site. As is standard practice in the art, chimeric nucleic acid molecules of the invention can be administered with an appropriate carrier.

The present invention involves direct administration of the combination of chimeric nucleic acid molecules of the invention to a subject. Alternative methods for administration include, but are not limited to, localized injection at a specific site, administration by implantable pump or continuous infusion, or liposomes.

The subject, so administered, is human, bovine, porcine, murine, equine, canine, feline, simian, ovine, piscine or avian.

The following examples are presented to illustrate the present invention and to assist one of ordinary skill in making and using the same. The examples are not intended in any way to otherwise limit the scope of the invention.

### EXAMPLES

### Example 1

The following Example provides a description of TLR3/TLR4-specific IRF3 mediated pathway that leads to an antiviral response.

Materials and Methods

Microarray and Clustering Analysis

B cell isolation, target preparation, and hybridization using Affymetrix Mu6500 microarrays were performed as described previously (Dadgostar, H., Zarnegar, B., Hoffman, A., Qin, X.-F., Truong, U., Rao, G., Baltimore, D., and Cheng, G. (2002), Proc. Natl. Acad. Sci. USA 99, 1497-1502). Differential expression data was analyzed by Affymetrix Microarray Suite 4.0 software. Average difference change values were then normalized and the genes were clustered by the uncentered correlation average linkage hierarchical clustering algorithm using Cluster. Data was then visualized as a dendogram using Treeview software (www.rana.Ibl.gov/EisenSoftware.htm).

Cell Culture and Reagents

Murine bone marrow-derived macrophages (BMMs) were differentiated from marrow from 6-10 week old C57B/6 mice as previously described (Chin, A. I., Dempsey, P. W., Beuhn, K., Miller, J. F., Xu, Y., and Cheng, G. (2002), Nature 416, 190-194). BMMs were maintained in 1× DMEM, 10% fetal bovine serum, 1% penicillin/streptomycin, and 30% L929 conditioned medium, and purity was assayed to be 94-99% CD11b⁺. The RAW 264.7 murine macrophage cell line (ATCC: TIB-71) was maintained in 1× DMEM with 10% fetal bovine serum and 1% penicillin/streptomycin. In order to study TLR activation, we used the following: 055-B5 E. coli LPS (Sigma), F-583 E. coli lipid A (Sigma), S. aureus peptidoglycan (Sigma), CpG oligonucleotides (Invitrogen), poly I:C (Pharmacia), and cycloheximide (Sigma). The dosage of poly I:C used was lowered from 10 μg/ml to 1 μg/ml for long term experiments to ensure viability of treated cells.

RNA Quantification

RNA was isolated for both Northern blotting and quantitative real-time PCR (Q-PCR) using a standard guanidium isothiocyanate method. Northern blotting was done as previously described (Lee, H., Dadgostar, H., Cheng, Q., Shu, J., and Cheng, G. (1999), Proc. Natl. Acad. Sci. USA 96, 9136-9141), and was hybridized using a RANTES cDNA fragment (IMAGE Clone: 832342, Research Genetics). For Q-PCR, RNA was quantitated and 2 μg of RNA was used to make cDNA templates using Superscript II (Invitrogen) according the the manufactuors instruction with either oligo-dT or random hexamer as primers. Q-PCR analyses was done using the iCycler thermocycler (Bio-Rad). Q-PCR was conducted in a final volume of 25 μL containing: Taq polymerase, 1× Taq buffer (Stratagene), 125 μM dNTP, SYBR™ Green I (Molecular Probes), and Fluoroscein (Bio-Rad), using oligo-dT cDNA or random hexamer cDNA as the PCR template. Amplification conditions were: 95° C. (3 min), 40 cycles of 95° C. (20 sec), 55° C. (30 sec), 72° C. (20 sec). The following primers were used to amplify a specific 100-120 bp fragment of the following genes:

| | |
|---|---|
| RANTES 5′: | GCCCACGTCAAGGAGTATTTCTA, |
| RANTES 3′: | ACACACTTGGCGGTTCCTTC, |
| Mx1 5′: | AAACCTGATCCGACTTCACTTCC, |
| Mx1 3′: | TGATCGTCTTCAAGGTTTCCTTGT, |
| IFI1 5′: | CCAGAGCATGGGAAAGAGGTT, |
| IFI1 3′: | CCGGACCTCTGATAGGAGACTG, |
| IFI-204 5′: | TTGGCTGCAATGGGTTCAT, |
| IFI-204 3′: | AGT GGGATATTCATTGGTTCGC, |
| IRF7 5′: | ACAGGGCGTTTTATCTTGCG, |
| IRF7 3′: | TCCAAGCTCCCGGCT AAGT, |
| IP-10 5′: | CCTGCCCACGTGTTGAGAT, |
| IP-10 3′: | TGATGGTCTTAGATTCCGGATTC, |
| ISG-15 5′: | CAGGACGGTCTTACCCTTT CC, |
| ISG-15 3′: | AGGCTCGCTGCAGTTCTGTAC, |
| IFIT1 5′: | GGCAGGAACAATGTGCAAGAA, |
| IFIT1 3′: | CTCAAATGTGGGCCTCAGTT, |
| 18S 5′: | CCGCGGTTCTATTTTGTTGGT, |
| 18S 3′: | CTCTAGCGGCGCAATACGA, |
| IFN-β 5′: | AGCTCCAAGAAAGGACGAACAT, |
| IFN-β 3′: | GCCCTGTAGGTGAGGTTGATCT, |
| IκBα 5′: | CTGCAGGCCACCAACTACAA, |
| IκBα 3′: | CAGCACCCAAAGTCACCAAGT, |
| Beta Actin 5′: | AGGTGTGCACCTTTTATTGGTCTCAA, |
| Beta Actin 3′: | TGTATGAAGGTTTGGTCTCCCT. |

Plasmid Constructions

The full-length and dominant-negative IRF3-expression plasmids were created by PCR amplifiction of IRF3 cDNA (IMAGE clone: 3666172) using either IRF3(1-420) 5′-CAGGACTGATCAACCATGGAAACCCCGAAACCGCGGATT-3′ or IRF3-DBD(133-420)5′-CAGGACATCCATGCACTCCCAGGAAAACCTACCGAA G-3′ in conjunction with the 3′ primer 5′-CAGGACGCGGCCGCGATATTCCAGT GGCCTGGAAGTC-3′. Fragments were cloned into the BglI/Not1 or BamHI/NotI sites of pEBB-puro. pCDNA3-IkBm-ER was constructed as described (Lee, H., Dadgostar, H., Cheng, Q., Shu, J., and Cheng, G. (1999), Proc. Natl. Acad. Sci. USA 96, 9136-9141). The −243 IP10 pCAT plasmid was a kind gift of Thomas A. Hamilton.

Transfections and CAT Assays

All transfections were done using Superfect™ (Qiagen) according to manufacturer's instructions. All plasmids were purified using Endo-free Maxiprep (Qiagen). Single cell clones for IκB and IRF3 constructs were selected for using 1 mg/ml G418 and 2.5 ng/ml puromycin, respectively. Chloramphenicol acetyl-transferase (CAT) assays were done as described elsewhere (Ohmori, Y., and Hamilton, T. A. (1993), J. Biol. Chem. 268, 6677-6688)

Immunoblotting, EMSA, and In Vitro Kinase Reactions

Cell fractionation and nuclear Western immunoblotting were done as described elsewhere (Lee, H., Dadgostar, H., Cheng, Q., Shu, J., and Cheng, G. (1999), Proc. Natl. Acad. Sci. USA 96, 9136-9141). Anti-IRF3 was obtained from Zymed, anti-USF-2 and anti-STAT1 from Santa Cruz Biotechnologies and antibodies specific to the phosphorylated forms of STAT1 and c-Jun were obtained from Cell Signaling Technologies. IFNβ blocking experiments employed an anti-IFNβ antibody (R&D Systems), anti-Type I IFNα/β (Access Biomedical, Inc), or non-specific Rabbit-IgG (Sigma) at final concentrations of 20 μg/ml. Cells were lysed in modified RIPA buffer, extracts were quantitated using either the Bradford assay reagent (Bio-Rad) or the BCA Protein Quantitation kit (Pierce), and 20 μg of protein were loaded in each lane and separated by SDS-PAGE. Gels were transferred to nitrocellulose filters and immunoblotted using the antibody manufacturers' recommended instructions. For detection of MHV68, rabbit anti-MHV68 was used as described by Wu, et. al. ((2001), J. Virol. 75, 9262-9273). To detect activation of JNK following TLR activation, in vitro kinase reactions were performed as previously described (Dadgostar, H., and Cheng, G. (2000), J. Biol. Chem. 275, 2539-2544). EMSA was done as previously described (Lee, H., Dadgostar, H., Cheng, Q., Shu, J., and Cheng, G. (1999), Proc. Natl. Acad. Sci. USA 96, 9136-9141).

Virus Production, Infection and Harvesting

Murine gammaherpesvirus 68 (MHV68) was produced and titered as previously described (Wu, T.-T., Tong, L., Rickabaugh, T., Speck, S., and Sun, R. (2001), J. Virol. 75, 9262-9273). For infection of macrophages, cells were simultaneously treated with PAMPs and infected with MHV68 at an MOI of 5. Following an incubation period of 48 hours cells were lysed in Laemmli buffer and 10% of total volume was subjected to SDS-PAGE, transferred to nitrocellulose, and MHV68 proteins detected by western blotting.

For NIH3T3 experiments, macrophages were first treated with PAMPs in the presence or absence of anti-Type I IFNα/β (Access Biomedical, Inc), or non-specific rabbit-IgG (Sigma) for a period of three hours. Conditioned medias were then collected and used to treat NIH3T3 cells for another three hours. Cells were then infected with MHV68 at an MOI of 1. Following an incubation period of 24 hours, cells were harvested and processed for viral content in an identical manner to the macrophages

Results

LPS Induces a Subset of Genes Previously Characterized as “Interferon-Regulated.”

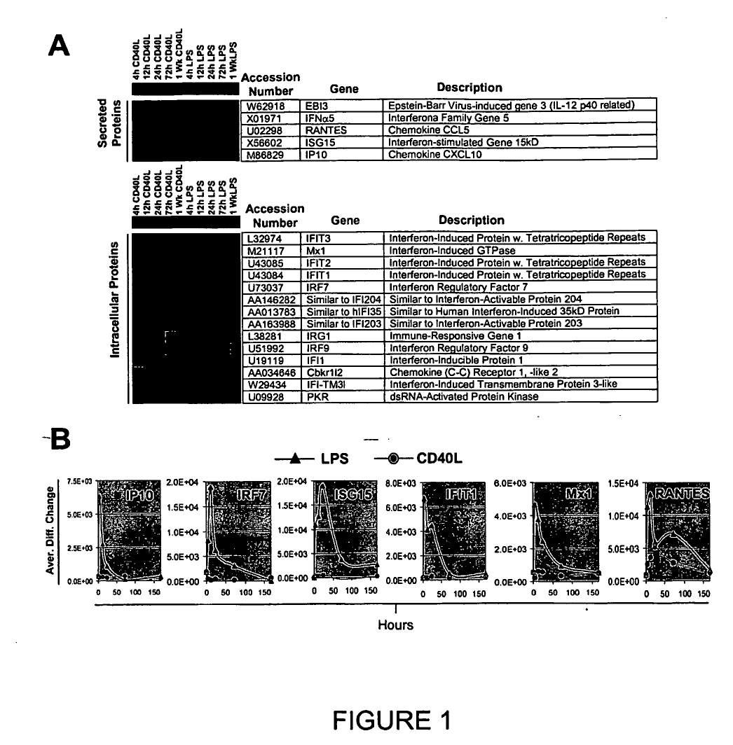

We have conducted a series of microarray experiments to determine gene expression patterns in murine B cells in response to activating stimuli such as LPS and CD40L. While CD40L specifically upregulates genes involved in cell-cell communication and germinal center formation (Dadgostar, H., Zarnegar, B., Hoffman, A., Qin, X.-F., Truong, U., Rao, G., Baltimore, D., and Cheng, G. (2002), Proc. Natl. Acad. Sci. USA 99, 1497-1502), hierarchical clustering and filtering of the microarray data also revealed a set of genes specifically induced by LPS, at least 19 of which have been previously classified as “interferon-regulated.” FIG. 1A depicts a partial list of LPS-specific genes using color-based gene expression changes of Affymetrix probe sets with matching accession numbers, gene names and descriptions. Genes were hierarchically clustered using average difference change values derived by comparing control samples (media 4h) with samples from cells treated with indicated stimulus. Line charts of selected genes (FIG. 1B) demonstrate similarities in kinetics of induction and LPS-specificity. Microarray studies on bone marrow-derived macrophages (BMM's) established that IFNβ mRNA was also specifically upregulated by LPS at two hours.

LPS-Primary Response Genes Exhibit TLR3/TLR4-Specifcity

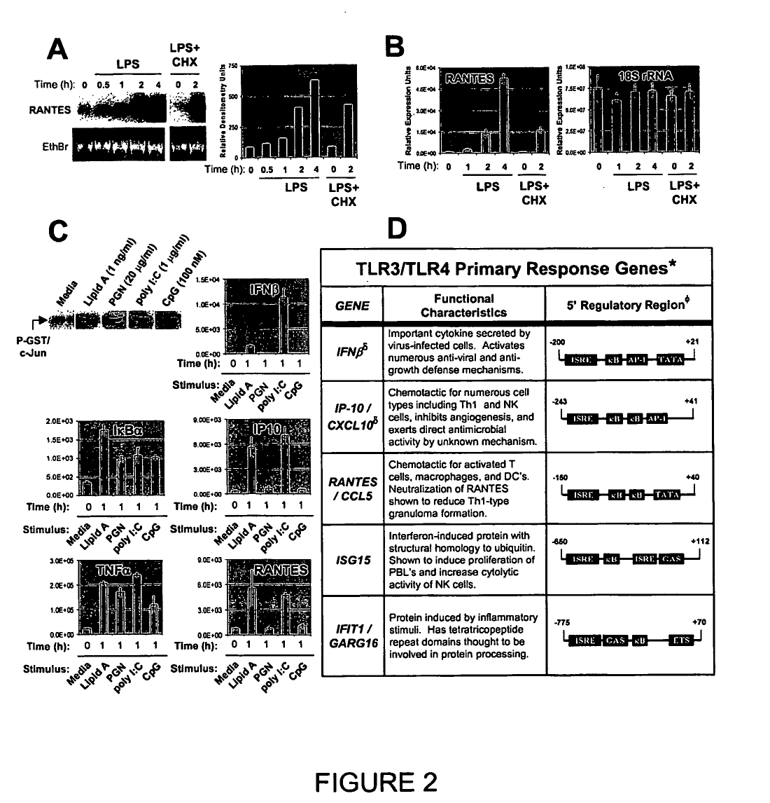

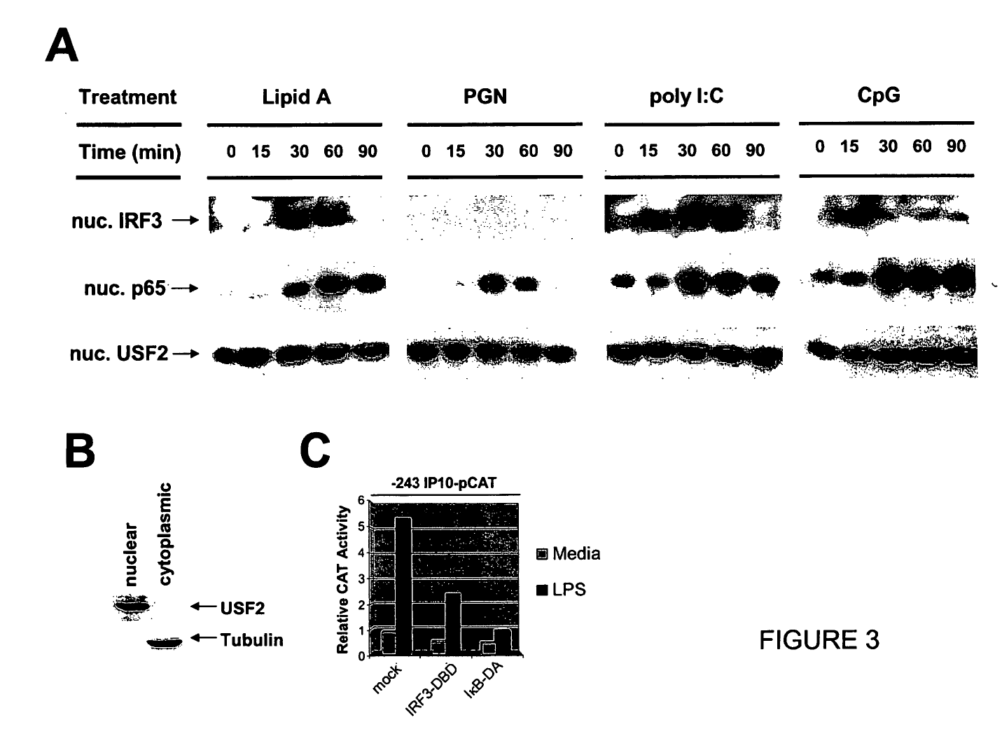

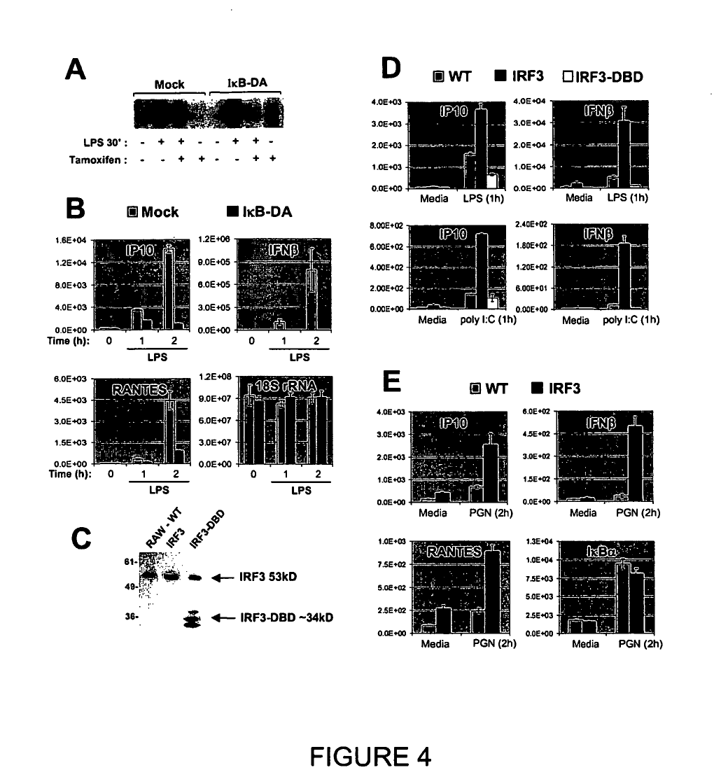

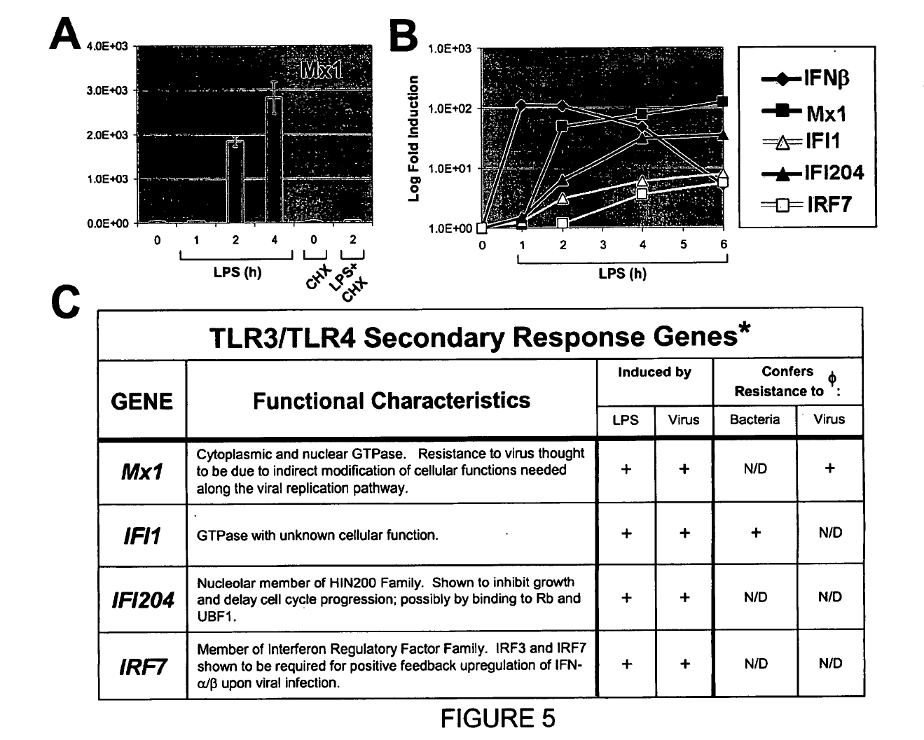

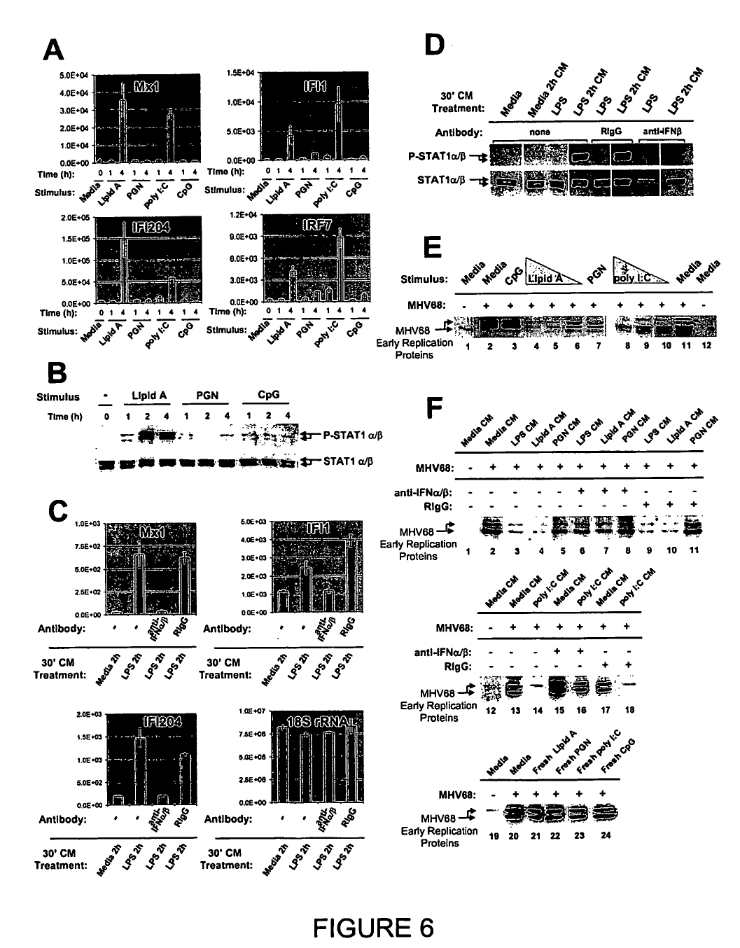

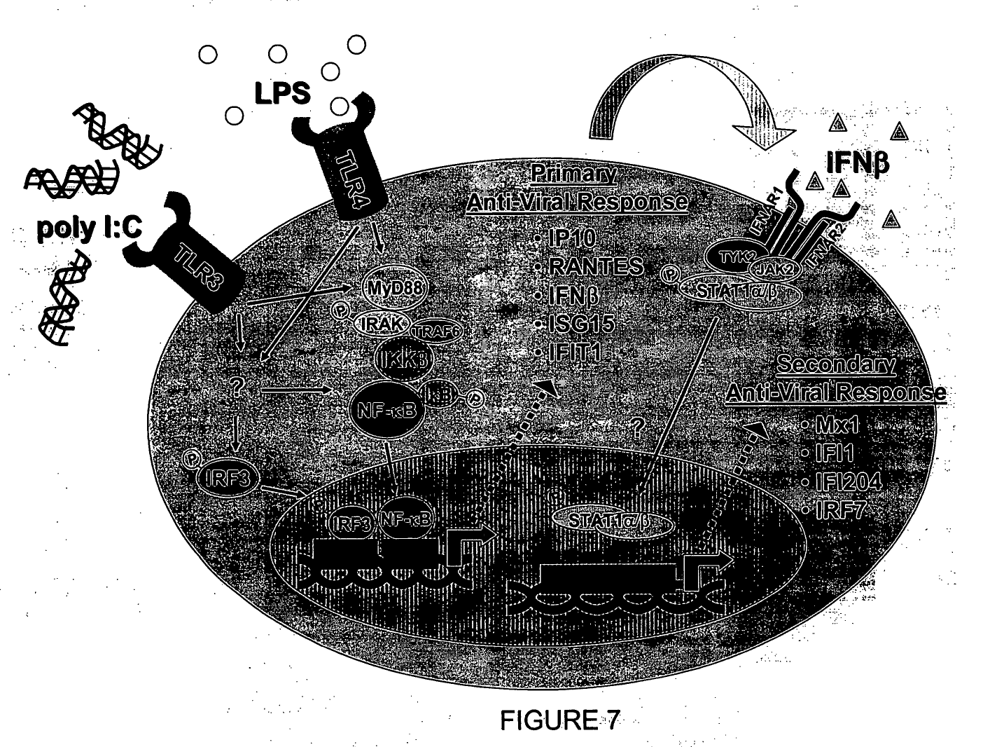

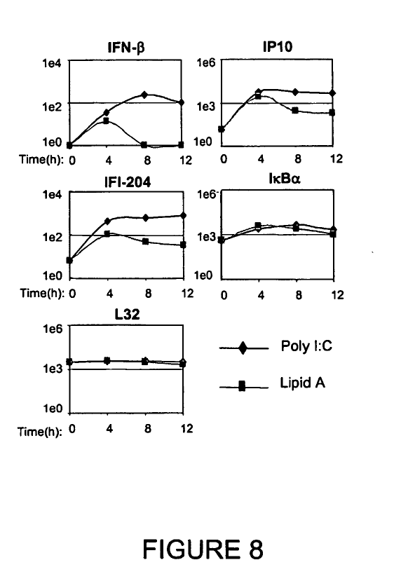

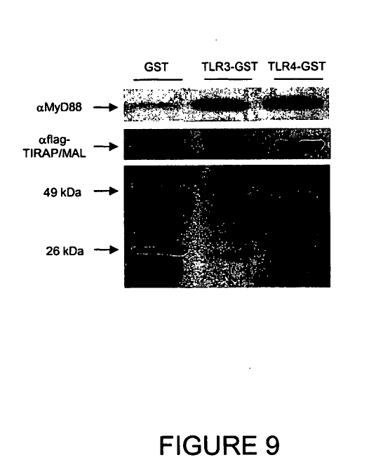

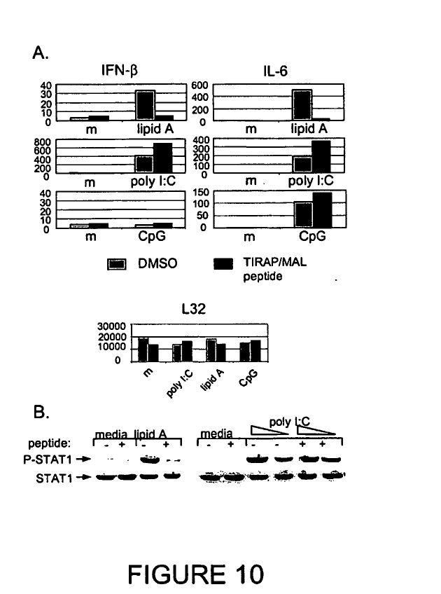

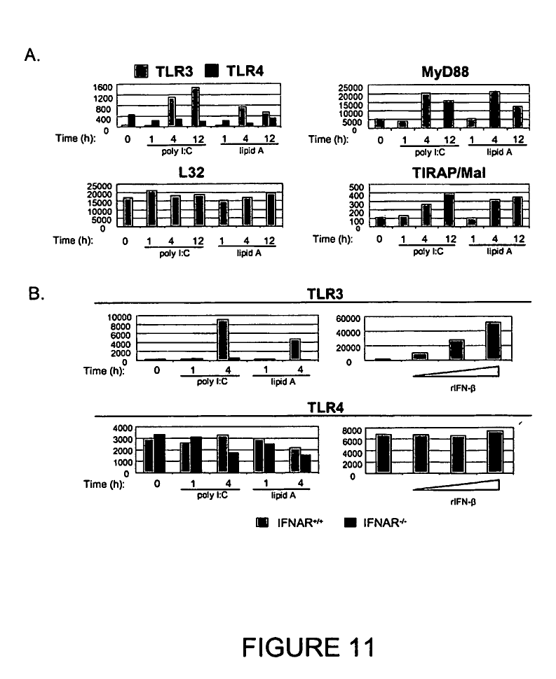

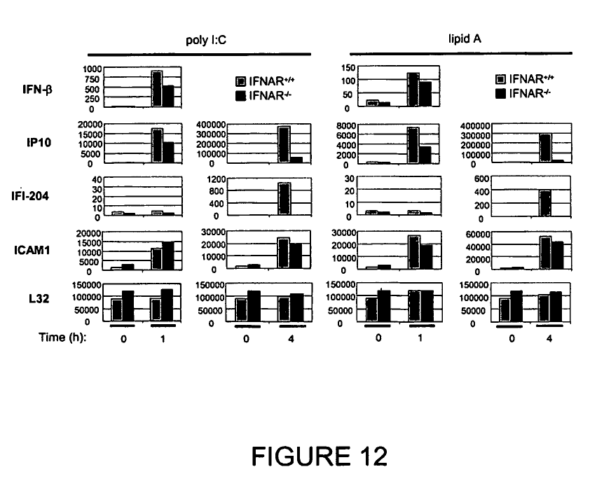

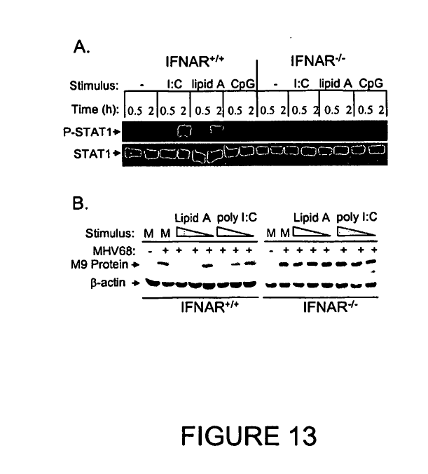

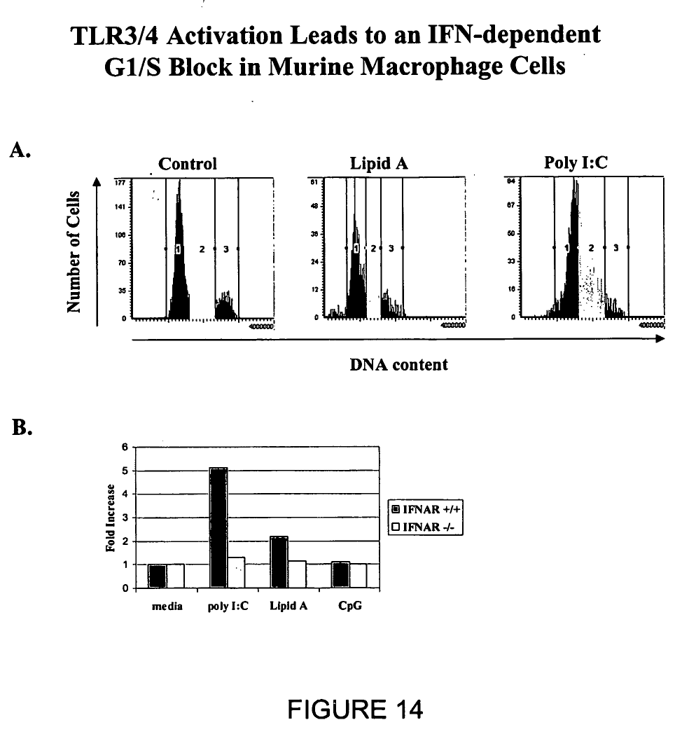

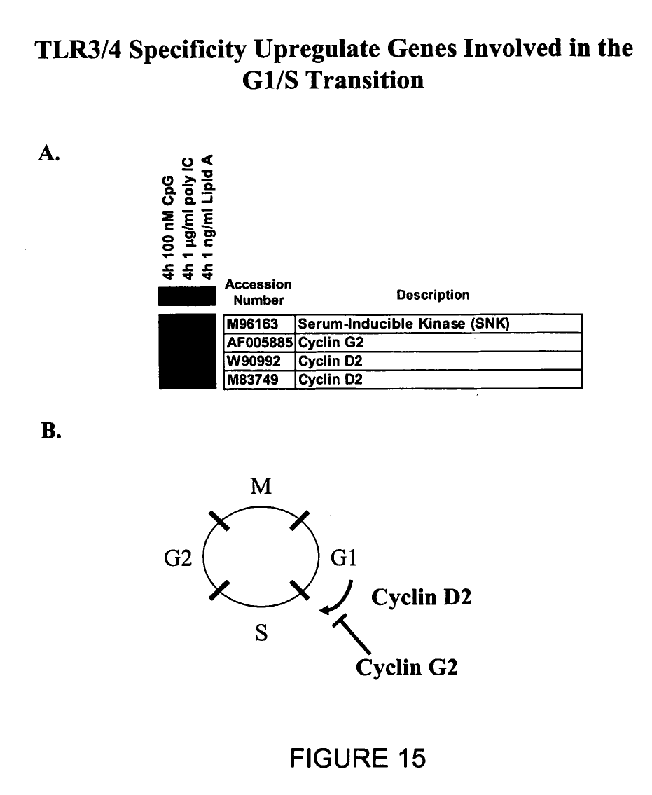

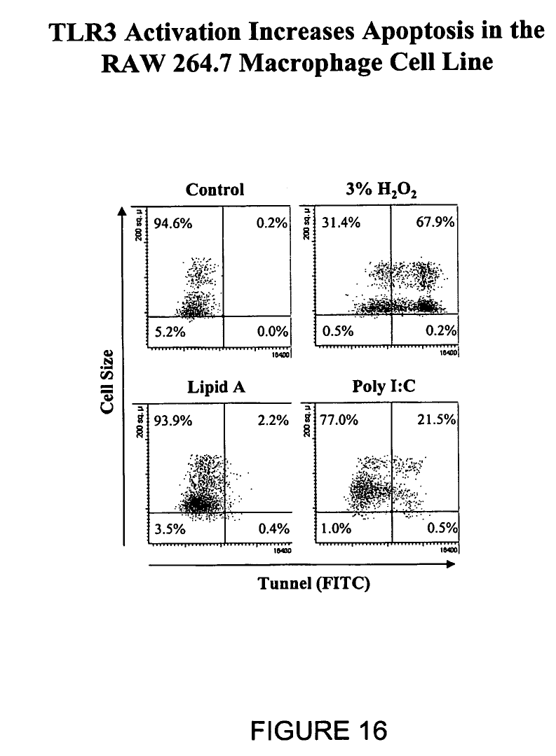

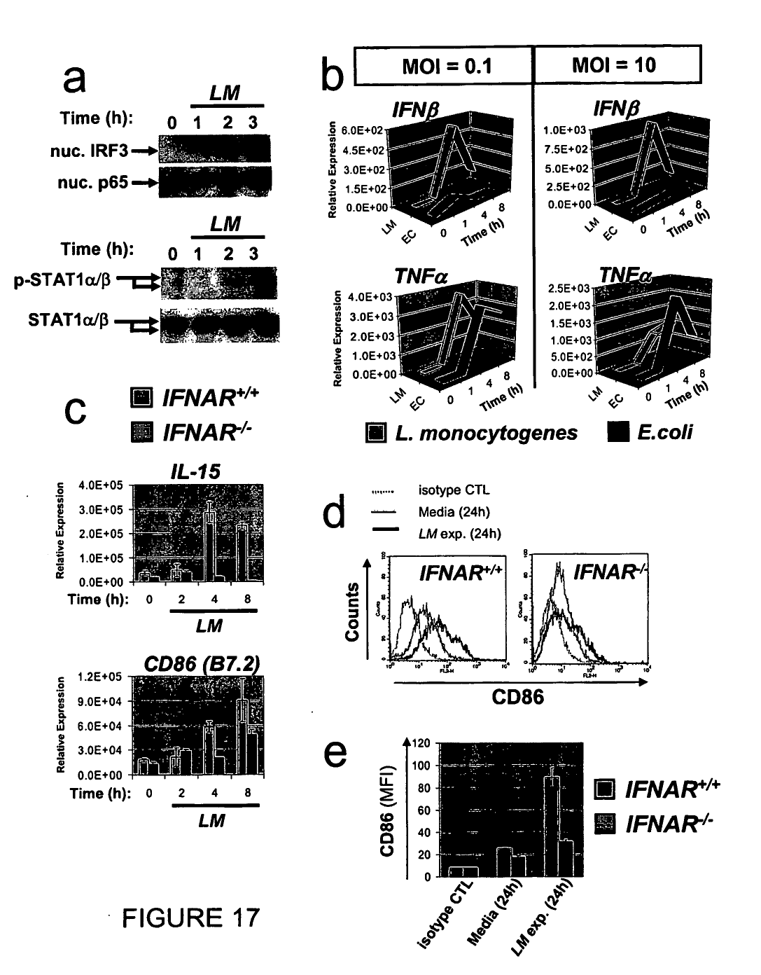

In order to understand the mechanism of selective gene activation by LPS, we conducted both Northern blot analysis and quantitative real-time PCR (Q-PCR), focusing on the macrophage cell type. As an example of our quantification methods, RANTES gene induction is shown using Northern blot analysis (FIG. 2A) and Q-PCR (FIG. 2B), which is used in all subsequent experiments. Throughout this report, the use of equivalent amounts of template in all Q-PCR reactions was controlled for through the measurement of 18S rRNA, except where noted. Cycloheximide treatment indicated that RANTES (FIGS. 2A, 2B), IP10, IFNβ, IFIT1 and ISG15 induction is the direct result of primary signal transduction and did not require new protein synthesis. Similar results were seen in B cells.

We then investigated the TLR-specificity of gene expression in BMMs using specific agonists for TLR4 (lipid A), TLR2 (peptioglycan), TLR3 (poly I:C) and TLR9 (CpG). In order to account for differences in binding affinity and receptor expression, the concentrations of TLR ligands used for stimulation were titrated to produce roughly equivalent activation of the JNK pathway as determined by GST-c-Jun in vitro kinase assay (FIG. 2C, upper left). Activation of NF-κB and the production of inflammatory cytokines are well-described for all known TLRs, and we found that our panel of TLR ligands induced both IκBα, a direct target of the NF-κB signaling pathway, and the inflammatory cytokine TNFα (FIG. 2C, middle left, lower left). However, only TLR3 or TLR4 stimulation led to the immediate early upregulation of IFNβ, IP10 and RANTES, while minimal gene induction was observed with TLR2 or TLR9-agonists (FIG. 2C, right panels). Interestingly, IFNβ was induced more potently by TLR3 than TLR4, while the chemokines IP10 and RANTES were induced to roughly equivalent levels by stimulation of either receptor. No gene induction was observed in response to lipid A in TLR4-null BMMs generated from C57BL/10ScCr mice that carry a null mutation in the TLR4 gene (Qureshi, S. T., Larivie' re, L., Leveque, G., Clermont, S., Moore, K. J., Gros, P., and Malo, D. (1999), J. Exp. Med. 189, 615-625).

A summary of five TLR3/TLR4-specific primary response genes—IP10, RANTES, IFNβ, ISG15 and IFIT1—is shown in FIG. 2D. These genes have been studied by other groups primarily in the context of viral infection and interferon stimulation (IP-10, (Cole, A. M., Ganz, T., Liese, A. M., Burdick, M. D., Liu, L., and Strieter, R. M. (2001), J. Immunol. 167, 623-627; Ohmori, Y., and Hamilton, T. A. (1993), J. Biol. Chem. 268, 6677-6688; Proost, P., Schutyser, E., Menten, P., Struyf, S., Wuyts, A., Opdenakker, G., Detheux, M., Parmentier, M., Durinx, C., Lambeir, A. M., et al. (2001), Blood 98, 3554-3561); RANTES, (Lin, R., Heylbroeck, C., Genin, P., Pitha, P. M., and Hiscott, J. (1999), Mol. Cell. Biol. 19, 959-966; Luther, S. A., and Cyster, J. G. (2001), Nat. Immunol. 2, 102-107; Wagner, L., Yang, O. O., Garcia-Zepeds, E. A., Ge, Y., Kalams, S. A., Walker, B. D., Pastemack, M. S., and Luster, A. D. (1998), Nature 391, 908-911); IFNβ, (Taniguchi, T., and Takaoka, A. (2002), Curr. Opin. Immunol. 14, 111-116); ISG15, (D'Cunha, J., Knight, E., Haas, A. L., Truitt, R. L., and Borden, E. C. (1996), Proc. Natl. Acad. Sci. USA 93, 211-215); IFIT1, (Guo, J., and Sen, G. C. (2000), J. Virol. 74, 1892-1899; Smith, J. B., and Herschman, H. R. (1996), Arch. Biochem. Biophys. 330, 290-300). To identify common elements that might mediate TLR3/TLR4-specific gene induction, we analyzed the gene promoters using the 5′ one kilobase sequence obtained from Celera proprietary murine genomic databases and TESS promoter analysis software (http://www.cbil.upenn.edu/tess/). The regulatory regions of all five genes showed high probability matches for ISRE and κB consensus sequences (Max. lg=>28.0) within a few hundred base pairs of the transcriptional start site (FIG. 2D). This indicated that these genes may be co-regulated by common activators which bind at these sites.

IRF3 and NF-κB are involved in TLR3/TLR4-Mediated Gene Activation.

Other groups studying models of viral infection have demonstrated binding of IRF3 to the ISRE consensus motifs in the promoters of IFNβ and RANTES (Lin, R., Heylbroeck, C., Genin, P., Pitha, P. M., and Hiscott, J. (1999), Mol. Cell. Biol. 19, 959-966; Wathelet, M. G., Lin, C. H., Parekh, B. S., Ronco, L. V., Howley, P. M., and Maniatis, T. (1999), Mol. Cell 1, 507-518). While LPS treatment can induce the nuclear translocation of IRF3 and induce ISRE binding in vitro at two hours of stimulation (Kawai, T., Takeuchi, O., Fujita, T., Inoue, J.-I., Mühlradt, P. F., Sato, S., Hoshino, K., and Akira, S. (2001), J. Immunol. 167, 5887-5894; Navarro, L., and David, M. (1999), J. Biol. Chem. 274, 35535-35538), it was recently reported that LPS does not increase IRF3 transactivational activity (Servant, M. J., ten Oever, B., LePage, C., Conti, L., Gessani, S., Julkunen, I., Lin, R., and Hiscott, J. (2001), J. Biol. Chem. 276, 355-363). As a result, the role of IRF3 in response to PAMP-induced gene expression remains in question. Our promoter analyses led us to investigate the activation of IRF3 and NF-κB by TLR stimuli, as these transcription factors bind to ISRE and κB consensus sites, respectively. We first confirmed that TLR3 and TLR4-agonists, but not TLR2 or TLR9-agonists, induced rapid nuclear translocation of IRF3 (FIG. 3A). However, unlike other reports, we found IRF3 to be activated within 15-30 minutes of treatment, and to be insensitive to cycloheximide treatment. In addition, stimulation of TLR3 could induce faster and more potent activation of IRF3 than TLR4, indicating further functional divergence between these two receptors. Similar results were seen with 1 μg/ml poly I:C. In contrast, we observed nuclear translocation of p65 in response to all TLR-agonists tested in BMMs (FIG. 3A) and RAW 264.7 macrophages. Purity of cellular fractions was monitored by immunoblotting nuclear and cytoplasmic fractions for the resident proteins USF2 and tubulin, respectively (FIG. 3B).

To determine whether IRF3 and NF-κB were involved in the LPS-induced transcriptional activity, we conducted chloramphenicol acetyl transferase (CAT) reporter assays in RAW 264.7 macrophages using the 5′-243 segment of the murine IP10 promoter. We co-transfected a dominant-negative mutant of IRF3 (IRF3-DBD) with a deletion of the N-terminal DNA-binding domain (133-420) (Lin, R., Mamane, Y., and Hiscott, J. (1999b), Mol. Cell. Biol. 19, 2465-2474) and IκB-DA (pCDNA3-IκBm-ER), a construct that encodes for a fusion protein of the estrogen receptor and an undegradable form of IκB that we have previously shown provides tamoxifen-inducible inhibition of NF-κB (Lee, H., Dadgostar, H., Cheng, Q., Shu, J., and Cheng, G. (1999), Proc. Natl. Acad. Sci. USA 96, 9136-9141). As shown in FIG. 3C, LPS treatment potently induced IP10 transactivation. However, this effect was inhibited by both IRF3-DBD and IκB-DA.

NF-κB Is Required for Upregulation of Primary Response Genes, While IRF3 Mediates TLR3/TLR4-Specifcity.

In order to further determine the role of NF-κB in LPS-induced gene expression, we transfected RAW cells with pCDNA3 (mock) or pCDNA3-IκBm-ER (IκB-DA). Single cell clones stably expressing these constructs were generated by G418 selection and were screened based on inhibition of LPS-induced nitric oxide production and lack of DNA binding activity by EMSA (FIG. 4A). FIG. 4B shows that LPS stimulation (100 ng/mL) of RAW-mock cells induced rapid upregulation of IP10, IFNβ and RANTES. However this was almost completely blocked in RAW-IκB-DA cells. These data provide evidence that NF-κB is required for the upregulation of LPS-primary response genes. We then created RAW cell lines stably expressing either full length IRF3 or IRF3-DBD (FIG. 4C). FIG. 4D shows Q-PCR analysis of gene expression in wild-type, IRF3 and IRF3-DBD RAW cells treated with LPS (100 ng/mL) (upper panels) or poly I:C (10 μg/mL) (lower panels). IRF3-overexpressing clones had both elevated basal and superinduction of several primary response genes within one hour of stimulation, while IRF3-DBD clones had inducible but reduced expression levels as compared to wild-type. Similar results were seen for ISG15 and IFIT1. Remarkably, overexpression of IRF3 conferred TLR2 responsiveness to TLR3/TLR4-specific genes (FIG. 4E), indicating that IRF3 may be sufficient for the specificity of gene expression observed. To demonstrate that IRF3 was not exerting non-specific effects, we analyzed IκBα gene induction, a direct target of the NF-κB signaling pathway. As shown in FIG. 4E (lower right), TLR2 stimulation with PGN induced similar levels of IκBα in RAW-WT and RAW-IRF3 cells lines. The integrity of Q-PCR analyses was controlled by β-actin mRNA levels. These data support the conclusion that while NF-κB is required for TLR-dependent gene activation, IRF3 is the principal component mediating the TLR3/TLR4-specificity of the primary response genes listed above.

Characterization of TLR3/TLR4-Secondary Response Genes

In the course of our gene expression analysis, we found that several genes initially screened were not induced until 2 h and were inhibited in the presence of cycloheximide. FIG. 5A shows an example of the induction pattern of one secondary response gene, Mx1; CHX treatment indicates that prior protein synthesis was required and that this gene is secondarily activated by a LPS-induced protein. Similar results were seen for IFI1, IFI204 and IRF7, and the overall kinetics of activation of these genes versus IFNβ (primary response) are shown in FIG. 5B. IFNβ is highly upregulated at 1-2 h, while secondary response genes are induced from 2-6 h. We focused on four genes—Mx1, IFI1, IFI204 and IRF7—whose gene products are thought to be involved in the development of innate immune responses (FIG. 5C) (Mx1, (Arnheiter, H., Skuntz, S., Noteborn, M., Chang, S., and Meier, E. (1990), Cell, 62, 51-61); IFI1, (Collazo, C. M., Yap, G. S., Sempowski, G. D., Lusby, K. C., Tessarollo, L., Woude, G. F. V., Sher, A., and Taylor, G. A. (2001), J. Exp. Med. 194, 181-187); IFI204, (Gariglio, M., Andrea, M. D., Lembo, M., Ravotto, M., Zappador, C., Valente, G., and Landolfo, S. (1998), J. Leukoc. Biol. 64, 608-614; Johnstone, R. W., and Trapani, J. A. (1999), Mol. Cell. Biol. 19, 5833-5838); IRF7, (Sato, M., Suemori, H., Hata, N., Asagiri, M., Ogasawara, K., Nakao, K., Nakaya, T., Katsuki, M., Noguchi, S., Tanaka, N., and Taniguchi, T. (2000), Immunity 13, 539-548; Taniguchi, T., Ogasawara, K., Takaoka, A., and Tanaka, N. (2001), Annu. Rev. Immunol. 19, 623-655).

TLR3/TLR4-Specific Production of IFNβ Activates Secondary Response Genes Involved in Host Defense

FIG. 6A demonstrates that TLR3 or TLR4-agonists, but not TLR2 or TLR9-agonists, could induce upregulation of the secondary response genes. We also found that activation of TLR4, but not TLR2 or TLR9, induced STAT1 phosphorylation (FIG. 6B), and that this effect could be blocked by treatment with cycloheximide. Type I IFNs (α/β) are known to induce STAT1α/β phosphoylation (Fu, X.-Y. (1992), Cell 70, 323-335; Schindler, C., Shuai, K., Prezioso, V. R., and Darnell, J. E. (1992), Science 257, 809-813), and while IFNβ is clearly a primary response gene (FIG. 2C), we found that no significant upregulation of IFNα subspecies mRNA occurred until 4 h as detected by Q-PCR analysis.

In order to investigate whether IFNβ was responsible for the activation of our subset of secondary response genes, we conducted experiments using the cell-free conditioned media (CM) of BMMs treated with TLR-agonists for two hours. As shown in FIG. 6C, treatment of fresh BMMs with LPS 2 h CM resulted in rapid activation of the LPS-secondary response genes within 30 minutes, as opposed to 2 h of treatment with LPS alone (FIG. 5B). The addition of anti-IFNα/β blocking antibodiesbut not non-specific rabbit IgG abolished this gene induction, demonstrating that IFNβ in the CM was responsible for this effect. Similar results were seen for IRF7. Notably, LPS 2 h CM, but not LPS alone, could also induce the rapid phosphorylation of STAT1 in 30 minutes, and this effect could be blocked by addition of anti-IFNβ (FIG. 6D). These data together demonstrate that the TLR4-specific upregulation of IFNβ can activate STAT1 and is responsible for the secondary upregulation of Mx1, IFI1, IFI204 and IRF7.

TLR3 and TLR4-Activation Inhibits MHV68 Replication

As some of the secondary response genes activated by TLR3 and TLR4 are known to play a role in viral resistance, we next sought to determine if these TLR ligands could directly inhibit the replication of murine gammaherpesvirus 68 (MHV68). BMMs were simultaneously infected with MHV68 (MOI=5) and treated with various TLR ligands (10, 1 or 0.1 ng/ml lipid A, 100 nM CpG, 10 μg/ml PGN or 1, 0.1, or 0.01 μg/ml poly I:C) for 48 hours, and replication of viral proteins was then assayed by western blot analysis. FIG. 6E demonstrates that either lipid A (lanes 4-6) or poly I:C (lanes 8-10) treatment could significantly inhibit MHV68 replication in a concentration dependent manner, while PGN had a smaller effect (lane 7), and CpG (lane 3) treatment was similar to the media control. During infections performed in the continuous presence of PGN, we repeatedly observed a minor inhibition in MHV68 replication. This was true whether BMMs were treated with 10 or 20 μg/ml PGN and the inhibition was always considerably weaker than that caused by either 1 ng/ml lipid A or 1 μg/ml poly I:C. These data indicate that among the TLRs tested, TLR3 and TLR4 are the strongest activators of genes that play a role in resistance to viral infection.

We have shown that TLR3 and TLR4 can specifically induce IFNβ and multiple downstream IFNβ response genes. However, the functional relevance of this signal and subsequent gene program were still undetermined. We therefore designed experiments in which we pretreated NIH3T3 cells (which are hyporesponsive to PAMP treatment) with the cell-free conditioned media (CM) from BMMs stimulated with PAMPs for three hours. We then assayed viral replication following 24 h infection MHV68. FIG. 6F shows that while media-treated control samples had significant amounts of viral protein (lane 2), only cells treated with CM from BMMs stimulated with TLR3 or TLR4 ligands were able to suppress viral replication (lanes 3, 4, and 14). Neither PGN CM nor direct treatment with PAMPs had a significant effect (lanes 5, and 21-24). Finally, inhibition of viral replication by TLR3 and TLR4 ligands was specifically abolished by addition of neutralizing antibodies to type I IFNα/β (lanes 6, 7, and 16). These data indicate that TLR3/TLR4-induced IFNβ mediates a functionally significant role in the innate immune response to viral infection.

Discussion

In this report, we have identified a specific subset of genes induced by stimulation of TLR3/TLR4 and demonstrated that IRF3 and NF-κK are key transcription factors responsible for this gene expression. While NF-κB was commonly activated by several TLRs, IRF3 was shown to direct the specific induction of a set of primary and secondary genes involved in host defense. Activation of the TLR3/TLR4 signaling pathway was also found to potently inhibit viral infection by MHV68 through the autocrine/paracrine production of IFNβ. Overall, we have described the signaling network that leads to the automatic and sequential activation of specific genes in response to dsRNA or LPS/Lipid A—a TLR3/TLR4-specific anti-viral gene program (FIG. 7). These data suggest that TLR3 and TLR4 have evolutionarily diverged from other members of the TLR family and can trigger important anti-viral responses through activation of IRF3.

Initially, our microarray data indicated that in B cells, LPS and CD40L activate many similar sets of genes for overlapping biological functions, such as cell survival, proliferation, metabolism and immunological isotype switching. CD40L specifically upregulated a subset of genes involved in cell adhesion, migration and germinal center formation (Dadgostar, H., Zamegar, B., Hoffman, A., Qin, X.-F., Truong, U., Rao, G., Baltimore, D., and Cheng, G. (2002), Proc. Natl. Acad. Sci. USA 99, 1497-1502), while LPS induced inflammatory cytokines (such as TNFα, IL-1β, and IL-6) and a subset of “interferon-associated” genes, as well as other poorly characterized genes with no previously described roles in TLR4 signaling. Our data further confirm results observed in other published LPS-gene expression studies. However, it is notable that the LPS-specific genes listed in FIG. 1A show remarkable overlap with genes upregulated by viral infection as indicated by viral gene expression studies (Geiss, G., Jin, G., Guo, J., Bumgarner, R., Katze, M. G., and Sen, G. C. (2001), J. Biol. Chem. 276, 30178-30182; Li, J., Peet, G.W., Balzarano, D., Li, X., Massa, P., Barton, R. W., and Marc, K. B. (2001), J. Biol. Chem. 276, 18579-18590; Suzuki, T., Hashimoto, S.-I., Toyoda, N., Nagai, S., Yamazaki, N., Dong, H.-Y., Sakai, J., Yamashita, T., Nukiwa, T., and Matsushima, K. (2000), Blood 96, 2584-2591; Zhu, H., Cong, J.-P., Mamtora, G., Gingeras, T., and Shenk, T. (1998), Proc. Natl. Acad. Sci. USA 95, 14470-14475). We and others have also confirmed that some LPS-primary response genes, such as IP10 and RANTES, are also secondarily upregulated by autocrine production of IFNβ (Ohmori, Y., and Hamilton, T. A. (200 1), J. Leukoc. Biol. 69, 598-604).

The specific activation of IRF3 by TLR3 and TLR4 led us to investigate gene expression with extensive titration of TLR-agonists. We found that increasing doses of PGN (25-50 μg/mL) could induce mild upregulation of type I interferon through an IRF3-independent mechanism, particularly at later time points. However, TLR3/TLR4-agonists at small doses (1 ng/mL LPS/lipid A or 1 μg/mL poly I:C) caused more than a 50-fold increase in gene expression by 2 h. In addition, TLR2 and TLR9-agonists were unable to induce detectable IRF3 nuclear translocation at any concentration tested. While the nuances of regulation of each individual gene are unique and outside the scope of this paper, the contribution of IRF3 to the enhanceosomes of some of these genes has been well documented in models of viral infection (Lin, R., Heylbroeck, C., Genin, P., Pitha, P. M., and Hiscott, J. (1999), Mol. Cell. Biol. 19, 959-966; Wathelet, M. G., Lin, C. H., Parekh, B. S., Ronco, L. V., Howley, P. M., and Maniatis, T. (1999), Mol. Cell 1, 507-18). Activation of IRF3 after viral infection has been shown to be the first step in activation of a “gene program” that includes a positive feedback loop of Type I IFNs and IRF family members (Taniguchi, T., Ogasawara, K., Takaoka, A., and Tanaka, N. (2001), Annu. Rev. Immunol. 19, 623-655). Interestingly, while the data presented here indicate that TLR3 and TLR4 activate gene expression by a similar mechanism at early time points, several lines of evidence suggest that even these receptors diverge with respect to their activation of innate anti-viral responses. Specifically, TLR3 induced a stronger activation of IRF3 (FIG. 3A), and this correlated with higher levels of IFNβ (FIG. 2C). Gene expression profiles from longer stimulations showed that while TLR4 induces IFNβ expression from one to four hours, TLR3 induces much higher levels of IFNβ with extended kinetics, with maximal levels at eight hours. This suggests that the TLR family of receptors have evolved to exert a stimulus-specific modulation of anti-viral responses while retaining pathways common to all TLRs that lead to production of proinflammatory genes such as TNFα (FIG. 2C).

While TLR4 can recognize some viral components, a critical question still remains—how and why do bacterial products such as LPS activate this pathway? The role of IRF3 or IFNβ in bacterial infection is not well understood. However, some recent reports have highlighted the ability of IFNβ to synergistically induce important components of the anti-microbial response such as iNOS and IFNβ (Jacobs, A. T., and Ignarro, L. J. (2001), J. Biol. Chem. 276, 47950-47957; Yaegashi, Y., Nielsen, P., Sing, A., Galanos, C., and Freudenberg, M. A. (1995), J. Exp. Med. 181, 953-960). On the other hand, another report found that Type I interferons are associated with increased susceptibility to bacterial infection by Mycobacterium tuberculosis (Manca, C., Tsenova, L., Bergtold, A., Freeman, S., Tovey, M., Musser, J. M., Barry, C. E., III, Freedman, V. H., and Kaplan, G. (2001), Proc. Natl. Acad. Sci. USA 98, 5752-5757).

While much is known about the biochemical events downstream in the TLR signaling pathways, evidence of increasing complexity between the individual receptors has led to a renewed interest in the biological role of the TLRs. Few studies have been able to conclusively prove increased susceptibility to a natural pathogen in TLR-deficient mice. However, the amazing detection capacities and evolutionary conservation of the TLRs strongly argue for an important functional role. Currently, it is unclear exactly how TLRs bind their ligands or cooperate with each other. It is possible that TLR3 and TLR4 may cooperate in the detection and response to certain viruses and may act separately or in conjunction with yet other TLRs to recognize other pathogens. Further work is certainly required to clarify this question.

Undoubtedly, activation of either TLR3 or TLR4 involves a much larger and more complex gene program than illustrated in this report. However, our findings show that these receptors can specifically activate signaling pathways that render cells more resistant to viral infection. TLR ligands can exert both immunostimulatory and toxic effects in vivo, and the data presented here identify distinct signaling pathways that lead to inflammatory or anti-viral responses. The identification of a specific gene program-activating “switch” that enhances innate anti-viral activity may provide promise for novel therapeutic treatments of viral infections. In addition, the development of pharmacological drugs that would allow manipulation of such a gene program might allow us to enhance the innate immunity in conditions where the adaptive immune system is compromised.

### Example 2

The following Example describes that TLR3 mediates a more potent antiviral response than TLR4.

Materials and Methods

Cell Culture and Reagents

Murine bone marrow-derived macrophages (BMMs) were differentiated from marrow as previously described (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251). A129 (IFNAR-1^{-/-}) (Muller, U., U. Steinhoff, L. F. L. Reis, S. Hemmi, J. Pavlovic, R. M. Zinkernagel, and M. Aguet. 1994. Functional role of type I and type II interferons in antiviral defense. Science 264:1918) and B6129SF2/J wild type control mice were obtained from B&K Universal Ltd. and Jackson Laboratories, respectively. C57/B6 mice were used for all experiments not involving the A129 mice (Jackson Laboratories). Specific TLR activation was achieved using F-583 (Rd mutant) E. coli lipid A for TLR4 (Sigma), CpG oligonucleotides for TLR9 (Invitrogen) and poly I:C for TLR3 (Pharmacia). For experiments employing the TIRAP/MAL inhibitory peptide (CN Biosciences), cells were pretreated for 1 hour with 20 μM peptide or DMSO alone. Cells were then stimulated with PAMPs in the presence of the inhibitory peptide. For experiments using murine rIFN-β (R&D Systems), wild-type macrophage cells were stimulated with 10, 100, or 1000 units. Viral infection and harvest was performed using MHV68 at an M.O.I. of 5 as previously described (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251).

mRNA Quantification

RNA was isolated by standard guanidium isothiocyanate methods. cDNA template for quantitative realtime PCR analysis was then synthesized and PCR was performed using the iCycler thermocycler (Bio-Rad) as previously described (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251). IFN-βIP10, IκBα and IFI-204 primers were the same as those previously described (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251). For other genes, the following primers were used:

| | |
|---|---|
| IL-6 Forward: | CACAGAGGATACCACTCCCAACA |
| and | |
| Reverse: | TCCACGATTTCCCAGAGAACA; |
| TLR3 Forward: | TCTGGAAACGCGCAAACC |
| and | |
| Reverse: | GCCGTTGGACTCTAAATTCAAGAT; |
| TLR4 Forward: | AGAAATTCCTGCAGTGGGTCA |
| and | |
| Reverse: | TCTCTACAGGTGTTGCACATGTCA; |
| TIRAP Forward: | CAGGCAGGCTCTGTTGAAGAA |
| and | |
| Reverse: | TGTGTGGCTGTCTGTGAACCA; |
| MyD88 Forward: | CATGGTGGTGGTTGTTTCTGAC |
| and | |
| Reverse: | TGGAGACAGGCTGAGTGCAA; |
| and | |
| ICAM1 Forward: | TGTCAGCCACTGCCTTGGTA |
| and | |
| Reverse: | CAGGATCTGGTCCGCTAGCT. |
| L32 Forward: | AAGCGAAACTGGCGGAAAC |
| and | |
| Reverse: | TAACCGATGTTGGGCATCAG. |

Plasmids and GST Pulldown Assays

A human TLR4 construct was generously provided by Dr. Robert Modlin at UCLA. ESTs containing the intracellular domain of hTLR3 and full length hMyD88 were obtained from Research Genetics. Each of the two constructs was used as a PCR template for amplification of the sequence corresponding to their respective intracellular domains. EcoRI and XhoI sites were engineered into the forward and reverse primer sequences, respectively, and used to ligate the PCR products into pGE1λT. The recombinant constructs were then transformed into Topp10 cells by electroporation. Following isopropyl β-D-thiogalactoside (IPTG)-induced expression, the cells were lysed in a Sarkosyl buffer (1% Sarkosyl, 100 mM EDTA, 1 mM DTT, in PBS) followed by sonication. The fusion proteins were then immobilized on glutathione beads (Sigma). The pCDNA3-2×Flag-mTIRAP/MAL construct was donated by Tapani Roni in Dr. Stephen Smale's laboratory at UCLA. The TIRAP/MAL and MyD88 constructs were overexpressed in 293T cells and lysed in IP lysis buffer (1% Triton X-100, 400 μM EDTA, 150 mM NaCl, 20 mM HEPES pH 7.2, 10 mM NaF and a protease inhibitor cocktail). The lysate was then incubated with the immobilized GST-TLR fusion proteins and interactions were detected by immunoblotting with an anti-flag monoclonal or anti-MyD88 polyclonal antibody.

Immunoblotting

For STAT1 immunoblotting, cells were lysed in modified RIPA buffer and 20 μg of protein were loaded per lane and separated by SDS-PAGE. Gels were transferred to nitrocellulose filters and immunoblotted using the antibody manufacturers' recommended instructions. Antibodies specific to the STAT1 or the phosphorylated forms of STAT1 were obtained from Cell Signaling Technologies and Santa Cruz Biotechnologies, respectively. The anti-MyD88 antibody was purchased from ProSci Incorporated. For detection of MHV68, equal amounts were loaded in each lane and analyzed by western blotting techniques using rabbit anti-M9. Blots were stripped and re-probed with anti-actin (Sigma) to verify equal loading.

Results

TLR3 is a More Potent Inducer of Antiviral Gene Expression than TLR4

We have previously shown that both TLR3 and TLR4 can induce a number of antiviral/IFN-β-inducible genes (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251). In order to compare the intensity and duration of expression of these genes following TLR3 versus TLR4 stimulation over an extended timecourse, we stimulated bone marrow-derived macrophage cells for up to twelve hours with either the TLR3 agonist poly I:C or the TLR4 agonist lipid A. Using quantitative realtime PCR technologies, we next assessed the expression levels of a number of antiviral genes throughout the timecourse. As seen in FIG. 8, IFN-β, IP10, and IFI-204 were all induced to higher levels and for extended periods of time by TLR3 compared to TLR4. Despite the increased induction of antiviral gene expression mediated by TLR3 relative to TLR4, both receptors induced IκBα mRNA to similar levels, albeit with slightly different kinetics (FIG. 8). The constitutively expressed ribosomal protein L32 was assayed to ensure equal cDNA loading. In FIG. 8 we used 10 μg/ml poly I:C for stimulations because it gave us comparable IκBα levels between both TLR3 and TLR4 stimulated cells. We also use 1 μg/ml poly I:C in later figures, which still results in higher TLR3-mediated antiviral gene induction compared to TLR4 (see FIG. 12), because it is less toxic to the cells.

TLR3 Can Directly Interact with MyD88 But Not with TIRAP/MAL

The receptor-proximal signaling complexes used by TLR3 and TLR4 to activate the antiviral gene program are relatively uncharacterized. We hypothesized that these receptors interact with distinct adaptor molecule-containing complexes which may contribute to the differences in signaling output observed in FIG. 8. MyD88 and TIRAP/MAL are both TIR-domain containing adaptor molecules that have been shown to directly bind to the cytoplasmic tail of TLR4 (Homg, T., G. M. Barton, and R. Medzhitov. 2001. TIRAP: an adapter molecule in the Toll signaling pathway. Nat. Immunol. 2:835; Fitzgerald, K. A., E. M. Pallson-McDermott, A. G. Bowie, C. A. Jeffries, A. S. Mansell, G. Brady, E. Brint, A. Dunne, P. Gray, M. T. Harte, et al. 2001. Mal (MyD88-adapter-like) is required for Toll-like receptor-4 signal transduction. Nature 413:78; Medzhitov, R., P. Preston-Hurlburt, E. Kopp, A. Stadlen, C. Chen, S. Ghosh, and C. A. Janeway, Jr. 1998. MyD88 is an adaptor protein in the hToll/IL-1 receptor family signaling pathways. Mol. Cell 2:253). However, these same experiments have not been performed with TLR3. To see if MyD88 or TIRAP/MAL is able to interact with TLR3, we performed GST pulldown assays. In order to conduct these experiments, we fused the complete intracellular domains of TLR3 and TLR4 to GST and immobilized the fusion proteins on glutathione agarose beads. Next, we attempted to capture overexpressed MyD88 or flag-TIRAP/MAL with the GST-TLR3 and GST-TLR4 beads. As expected, MyD88 and TIRAP/MAL bound to TLR4. The TLR3 intracellular domain was also able to associate with MyD88. However, we found that TLR3 did not interact with TIRAP/MAL (FIG. 9). These data strongly suggest that the receptor-proximal signaling complex directly engaged by TLR3 differs compositionally from the complex engaged by TLR4.

The TIRAP/MAL Inhibitory Peptide is Able to Block TLR4 But Not TLR3 Signaling

Although knockout studies have suggested that both MyD88 and TIRAP/MAL are dispensable for induction of IFN-β by TLR3 and TLR4dominant negative TIRAP/MAL has been shown to prevent TLR4 but not TLR3 signaling through IRF3 (Shinobu, N., T. Iwamura, M. Yoneyama, K. Yamaguchi, W. Suhara, Y. Fukuhara, F. Amano, and T. Fujita. 2002. Involvement of TIRAP/MAL in signaling for the activation of interferon regulatory factor 3 by lipopolysaccharide. FEBS Lett. 51 7:251; Kawai, T., O. Adachi, T. Ogawa, K. Takeda, and S. Akira. 1999. Unresponsiveness of MyD88-deficient mice to endotoxin. Immunity 11:115; Yamamoto, M., S. Sato, H. Hemmi, H. Sanjo, S. Uematsu, T. Kaisho, K. Hoshino, O. Takeuchi, M. Kobayashi, T. Fujita, et al. 2002. Essential role for TIRAP in activation of the signalling cascade shared by TLR2 and TLR4. Nature 420:324; Medzhitov, R., P. Preston-Hurlburt, E. Kopp, A. Stadlen, C. Chen, S. Ghosh, and C. A. Janeway, Jr. 1998. MyD88 is an adaptor protein in the hToll/IL-1 receptor family signaling pathways. Mol. Cell 2:253). In addition, a cell perrneable TIRAP/MAL-inhibitory peptide has been shown to block TLR4 mediated induction of an IFN-β-specific reporter construct in RAW 264.7 cells (Toshchakov, V., B. W. Jones, P.-Y. Perera, K. Thomas, M. J. Cody, S. Zhang, B. R. G. Williams, J. Major, T. A. Hamilton, M. J. Fenton, and S. N. Vogel. 2002. TLR4, but not TLR2, mediates IFN-β-induced STAT1α/β-dependent gene expression in macrophages. Nat. Immunol. 3:392). However, this inhibitory peptide has not been used to study TLR4 signaling in primary macrophage cells, nor has its affects on TLR3 signaling been addressed.

Because knockout studies leave open the possibility for redundancy, we decided to assess whether the TIRAP/MAL peptide could block TLR3 or TLR4 induction of IFN-β gene expression and activation of STAT1 following primary macrophage treatment with either TLR3 or TLR4 ligands. Results from these experiments show that the TIRAP/MAL peptide abrogated TLR4-mediated expression of IFN-β and STAT1 activation in primary macrophage cells. These findings corroborate peptide studies using macrophage cell lines, but disagree with TIRAP/MAL knockout results (Toshchakov, V., B. W. Jones, P.-Y. Perera, K. Thomas, M. J. Cody, S. Zhang, B. R. G. Williams, J. Major, T. A. Hamilton, M. J. Fenton, and S. N. Vogel. 2002. TLR4, but not TLR2, mediates IFN-β-induced STAT1α/β-dependent gene expression in macrophages. Nat. Immunol. 3:392; Horng, T., G. M. Barton, R. A. Flavell, and R. Medzhitov. 2002. The adaptor molecule TIRAP provides signalling specificity for Toll-like receptors. Nature 420:329; Yamamoto, M., S. Sato, H. Hemmi, H. Sanjo, S. Uematsu, T. Kaisho, K. Hoshino, O. Takeuchi, M. Kobayashi, T. Fujita, et al. 2002. Essential role for TIRAP in activation of the signalling cascade shared by TLR2 and TLR4. Nature 420:324) (FIGS. 43A and 43B). In contrast to its effect on TLR4 signaling, the peptide was completely unable to block TLR3 induced expression of IFN-β and IL-6, which is in complete agreement with all previous studies. (FIG. 10A). Likewise, STAT1 was still activated in cells stimulated with poly I:C in the presence of the TIRAP/MAL peptide (FIG. 10B). Even at lower concentrations of poly I:C stimulation (FIG. 10B) or higher concentrations of the TIRAP/MAL peptide, the inhibitor was still incapable of blocking STAT1 activation via TLR3 signaling. Poly I:C has been shown to weakly induce proinflammatory cytokine production in TLR3 deficient mice, while poly I:C induced IFN-β induction appears to be TLR3-dependent (Alexopoulou, L., A. C. Holt, R. Medzhitov, and R. A. Flavell. 2001. Recognition of double-stranded RNA and activation of NF-κK by Toll-like receptor 3. Nature 413:732). Although we cannot exclude the possibility that poly I:C may signal through alternative receptors other than TLR3, our studies show that the peptide is incapable of reducing poly I:C mediated gene expression and that TIRAP/MAL cannot bind to the cytoplasmic tail of TLR3. These data strongly suggest that TLR3 does not utilize TIRAP/MAL for signaling.

We found that the TIRAP/MAL peptide inhibited IL-6 expression following TLR4 ligation, which is consistent with TIRAP/MAL knockout data (Horng, T., G. M. Barton, R. A. Flavell, and R. Medzhitov. 2002. The adaptor molecule TIRAP provides signalling specificity for Toll-like receptors. Nature 420:329; Yamamoto, M., S. Sato, H. Hemmi, H. Sanjo, S. Uematsu, T. Kaisho, K. Hoshino, O. Takeuchi, M. Kobayashi, T. Fujita, et al. 2002. Essential role for TIRAP in activation of the signalling cascade shared by TLR2 and TLR4. Nature 420:324). One possible explanation for this would be that the TIRAP/MAL peptide not only affected TIRAP/MAL but also interfered with MyD88 function. TLR9 signaling has previously been shown to be completely dependent on MyD88. We therefore used TLR9-mediated IL-6 activation as a readout to determine whether the TIRAP/MAL peptide could specifically interrupt MyD88 signaling (Horng, T., G. M. Barton, and R. Medzhitov. 2001. TIRAP: an adapter molecule in the Toll signaling pathway. Nat. Immunol. 2:835). Consistent with previous reports using dendritic cells, we found that CpG-induced IL-6 expression in macrophages was not affected by the TIRAP/MAL peptide (FIG. 10A). Thus, the TIRAP/MAL peptide does not specifically interfere with MyD88 signaling. These results show that the TIRAP/MAL inhibitory peptide can disrupt TLR4, but not TLR3 or TLR9, signaling in primary macrophage cells. Furthermore, since the peptide appears to inhibit both MyD88-dependent and -independent signaling events following TLR4 stimulation, the peptide may disrupt the entire TLR4-proximal signaling complex.

TLR3 and TLR4 Ligands Induce Expression of TLR3, MyD88, and TIRAP/MAL

Thus far our data suggest that TLR3 and TLR4 signal via unique adaptor molecule-containing complexes. We next wanted to see if either TLR3 or TLR4 was capable of transcriptionally inducing molecules involved in eliciting early signaling events, which may explain why TLR3 can sustain and enhance antiviral gene expression to a greater degree than TLR4. To address this issue, we stimulated primary macrophage cells with poly I:C or lipid A for up to twelve hours. By four hours, treatment of macrophages with TLR3 or TLR4 agonists caused the induction of TLR3, but not TLR4, mRNA production (FIG. 11A). We also observed that TLR9 signaling, which does not induce IFN-β in primary macrophage cells, was incapable of inducing TLR3 expression . In FIG. 11A we show that both TLR3 and TLR4 agonists can induce the expression of MyD88 as well as TIRAP/MAL. Taken together, these data suggest that TLR3 is able to prolong and enhance its induction of antiviral genes by rapidly upregulating the expression of additional TLR3.

TLR3 and TLR4 Induce TLR3 Expression Through IFN-β

We have shown that both TLR3 and TLR4 utilize the IRF3 transcription factor to induce IFN-β gene expression (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251). Once secreted from the cell, IFN-β is believed to act in an autocrine/paracrine manner leading to STAT1 activation and secondary antiviral gene induction (Toshchakov, V., B. W. Jones, P.-Y. Perera, K. Thomas, M. J. Cody, S. Zhang, B. R. G. Williams, J. Major, T. A. Hamilton, M. J. Fenton, and S. N. Vogel. 2002. TLR4, but not TLR2, mediates IFN-β-induced STAT1α/β-dependent gene expression in macrophages. Nat. Immunol. 3:392.; Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251; Ohmori, Y., and T. A. Hamilton. 2001. Requirement for STAT1 in LPS-induced gene expression in macrophages. J. Leukocyte Biol. 69:598). Due to the fact that both TLR3 and TLR4 can induce TLR3 expression, and because this induction takes place after IFN-β production has begun, we investigated whether TLR3 expression was induced by IFN-β. Using cells deficient in the IFN-α/β Receptor (IFNAR), we show in FIG. 11B that treatment of cells with TLR3 or TLR4 agonists does not cause the induction of TLR3 expression in the absence of IFNAR. Furthermore, stimulating primary macrophage cells with recombinant IFN-β resulted in a dose dependent upregulation of TLR3 mRNA (FIG. 11B). TLR4 mRNA levels were relatively unaltered in the IFNAR deficient cells or by induction of wild type cells with rIFN-β (FIG. 11B). These data indicate that TLR3 and TLR4 can potently induce TLR3, but not TLR4, expression through IFN-β production.

TLR3 and TLR4 Induce Both IFN-β Enhanced and IFN-β Dependent Antiviral Genes.

We have previously characterized TLR3 and TLR4 antiviral gene induction as either primary or secondary based upon sensitivity to cyclohexamide (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251). Our previous data suggested that primary genes are induced in the absence of novel protein synthesis, while secondary genes require the initial expression of IFN-β. To further characterize these primary and secondary genes, we induced both wild-type and IFNAR deficient cells with either poly I:C or lipid A and assessed antiviral gene expression at one and four hours. As seen in FIG. 12, IFN-β and IP10 (both primary genes) are induced by one hour in both wild type and IFNAR knockout macrophage cells, while IFI-204 (a secondary gene) remained at basal levels. By four hours IP10 expression was significantly enhanced in the wild type cells, but remained relatively unchanged in the IFNAR knockout cells. These data indicate that the primary expression of IP10 is enhanced by the IFN-β positive feedback loop. The secondary gene, IFI-204, was induced to high levels by four hours, yet was not detectable in the IFNAR knockout cells at the same time point. Similar results were also obtained for the primary and secondary genes RANTES and Mx1, respectively. As an induction control, ICAM1 mRNA was elevated by one hour and remained high by four hours in both wild-type and knockout cells stimulated with poly I:C or lipid A. As shown in FIG. 11B, TLR3 is part of the secondary gene subset.

The IFN-α/β Receptor (IFNAR) is Required for Both TLR3 and TLR4 Activation of STAT1 and Resistance to MHV68 Infection.

In mice deficient in the IFN-α/βreceptor (IFNAR), STAT1 activation has been shown to be blocked in macrophage cells stimulated with LPS (Ohmori, Y., and T. A. Hamilton. 2001. Requirement for STAT1 in LPS-induced gene expression in macrophages. J. Leukocyte Biol. 69:598). Although blocking antibody studies have suggested that IFN-β is also essential for poly I:C-induced STAT1 activation, these studies have not been conducted to date in primary macrophage cells deficient in IFNAR (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251). To address this issue we stimulated IFNAR^{-/-} BMMs with lipid A and poly I:C and assayed for STAT1 phosphorylation. In FIG. 13A, we show that like TLR4, TLR3-mediated STAT1 activation was also abolished in IFNAR^{-/-} macrophage cells. The TLR9 agonist CpG, which fails to induce IFN-β in primary macrophage cells, was used as a negative control.

Blocking antibody and conditioned media experiments have suggested that TLR3- and TLR4-mediated viral resistance is IFN-β dependent (Doyle, S. E., S. A. Vaidya, R. O'Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251). Using the MHV68 protein M9 as a readout for viral load, we show in FIG. 13B that the antiviral activity of macrophage cells infected with MHV68 was abolished in the absence of IFNAR despite co-treatment with either lipid A or poly I:C. Thus, IFNAR-mediated upregulation of secondary response genes, such as IFI-204, and enhancement of primary genes, such as IP10, is essential for antiviral activity. In fact, MHV68 protein synthesis was enhanced in IFNAR^{-/-} versus wild-type cells under all conditions tested. These data provide genetic evidence that the IFN-β autocrine/paracrine loop is essential for induction of the TLR3- and TLR4-specific antiviral gene program.

Discussion:

Our data suggest that although both TLR3 and TLR4 induce antiviral gene expression, TLR3 is better suited than TLR4 to activate this program. We show that TLR3 is able to induce higher levels of IFN-β which is most likely a result of using a different signaling complex that can more strongly activate IRF3 than TLR4. In addition, TLR3 is also able to enhance its own expression (via an IFN-β-mediated positive feedback loop), thereby promoting an even stronger antiviral response. Viral infection or IFN-α stimulation of human macrophage cells has also been shown to induce TLR3 transcription (Miettinen, M., T. Sareneva, I. Julkunen, and S. Matikainen. 2001. IFNs activate Toll-like receptor gene expression in viral infections. Genes Immun. 2:349). Collectively, the data argue that while both TLR3 and TLR4 have been evolutionarily selected to induce antiviral gene expression, TLR3 seems to be even more specialized than TLR4 to initiate antiviral responses and is specifically upregulated when a virus is detected.

Sequence analysis of the BB loop region (found in the TIR domain) reveals significant homology between the BB loop domains of TLR3, TLR4, MyD88 and TIRAP/MAL. The importance of this region in TLR signaling is exemplified by C3H/HeJ mice which contain a P712H mutation in the BB loop that renders these mice incapable of signaling via TLR4 (Qureshi, S. T., L. Larivie're, G. Leveque, S. Clermont, K. J. Moore, P. Gros, and D. Malo. 1999. Endotoxin-tolerant mice have mutations in Toll-like receptor 4 (Tlr4). J. Exp. Med. 189:615). A P125H mutation in the homologous region of TIRAP/MAL prevents association with TLR4 (Fitzgerald, K. A., E. M. Pallson-McDermott, A. G. Bowie, C. A. Jeffries, A. S. Mansell, G. Brady, E. Brint, A. Dunne, P. Gray, M. T. Harte, et al. 2001. Mal (MyD88-adapter-like) is required for Toll-like receptor-4 signal transduction. Nature 413:78). Interestingly, TLR3 naturally contains an alanine instead of a proline at this same BB loop position, which may explain why we do not detect TIRAP/MAL interacting with TLR3.

The fact that TLR4 can still activate IRF3 in primary macrophage cells deficient in TIRAP/MAL, but dominant negative TIRAP/MAL and the TIRAP/MAL inhibitory peptide can block TLR4 mediated IFN-β expression presents a conflicting situation regarding the actual role of TIRAP/MAL in TLR4-mediated antiviral gene induction (Shinobu, N., T. Iwamura, M. Yoneyama, K. Yamaguchi, W. Suhara, Y. Fukuhara, F. Amano, and T. Fujita. 2002. Involvement of TIRAP/MAL in signaling for the activation of interferon regulatory factor 3 by lipopolysaccharide. FEBS Lett. 517:251; Toshchakov, V., B. W. Jones, P.-Y. Perera, K. Thomas, M. J. Cody, S. Zhang, B. R. G. Williams, J. Major, T. A. Hamilton, M. J. Fenton, and S. N. Vogel. 2002. TLR4, but not TLR2, mediates IFN-β-induced STAT1α/β-dependent gene expression in macrophages. Nat. Immunol. 3:392; Yamamoto, M., S. Sato, H. Hemmi, H. Sanjo, S. Uematsu, T. Kaisho, K. Hoshino, O. Takeuchi, M. Kobayashi, T. Fujita, et al. 2002. Essential role for TIRAP in activation of the signalling cascade shared by TLR2 and TLR4. Nature 420:324). As mentioned, the use of TIRAP/MAL-deficient cells leaves open the possibility that a redundant molecule may replace TIRAP/MAL in the TLR4-specific receptor-proximal signaling complex. On the other hand, the inhibitory peptide and the dominant negative form of TIRAP/MAL may nonspecifically interfere with other TIR containing molecules. Thus, both experimental methods have possible defects that may lead to the conflicting results observed. In spite of this, it is apparent that TIRAP/MAL can interact with TLR4 and is involved in certain aspects of TLR4 signaling. However, this does not appear to be the case for TLR3. The results presented in this manuscript strongly suggest that TIRAP/MAL is unable to interact with TLR3 and is also not involved in TLR3-activated signal transduction.

It is apparent that both TLR3 and TLR4 can activate similar IFN-β-mediated antiviral gene programs, and that IFN-β is a key mediator of these responses. Our results clearly demonstrate that antiviral genes induced by TLR3 and TLR4 fall into two distinct categories; primary, which are insensitive to cycloheximide and are initially still induced early (by 1 h) in the absence of IFN-β, yet are greatly enhanced by the IFN-β positive feedback loop; and secondary, which are not induced in the presence of cycloheximide or until IFN-β is produced and feeds back to signal through its receptor, IFNAR (by approximately 2-4 h). Our previous data have suggested that a key difference between primary and secondary gene induction is that primary genes appear to be transactivated directly by IRF3, in addition to NFκB, following TLR3 or TLR4 ligand engagement (Doyle, S. E., S. A. Vaidya, R. O° Connell, H. Dadgostar, P. W. Dempsey, T.-T. Wu, G. Rao, R. Sun, M. E. Haberland, R. L. Modlin, and G. Cheng. 2002. IRF3 mediates a TLR3/TLR4-specific antiviral gene program. Immunity 17:251).

It remains unresolved how IRF3 becomes activated following TLR3 or TLR4 receptor stimulation. It is also very likely that other TLRs may contain their own unique signaling pathways involving as yet unidentified signaling mediators. In addition to MyD88 and TIRAP/MAL, only a few other proteins have been shown to interact directly with the intracellular domains of TLRs, including the Rho GTPase Rac-1, PI3K and Tollip (Arbibe, L., J.-P. Mira, N. Teusch, L. Kline, M. Guha, N. Mackman, P. J. Godowski, R. J. Ulevitch, and U. G. Knaus. 2000. Toll-like receptor 2-mediated NF-κB activation requires a Rac1-dependent pathway. Nat. Immunol. 1:533; Bums, K., J. Clatworthy, L. Martin, F. Martinon, C. Plumpton, B. Maschera, A. Lewis, K. Ray, J. Tschopp, and F. Volpe. 2000. Tollip, a new component of the IL-IRI pathway, links IRAK to the IL-1 receptor. Nat. Cell Biol. 2:346) The EST database currently contains a large number of TIR domain-containing sequences. It may be that one or more of these proteins plays a role in mediating the activation of IRF3 downstream of TLR3. By continuing to characterize these putative and established TLR-interacting adaptor molecules, the signaling and functional specificities between the different TLRs will surely become more clearly understood.

### Example 3

The following Example describes that TLR3/4 activation leads to an IFN-dependent G1/S block in murine macrophage cells.

The RAW 264.7 murine macrophage cell line was treated for two 24 h intervals with media alone (control), 10 ng/ml Lipid A (Lipid A) or 10 mg/ml poly I:C. Cells were then fixed and permeablized and treated with the DNA-intercalating dye, DAPI (pharmingen). DNA content was then measured by laser scanner cytometry (LSC). The cell cycle is divided into G1 (red), S-phase (yellow) and G2/M (blue) as shown in FIG. 14A.

Primary bone marrow-derived macrophages from wild-type (IFNAR+/+) and IFNα/β receptor knockout (IFNAR−/−) mice were treated as indicated in (FIG. 14A). The cells were then pulsed with BrdU for two hours to label S-phase cells. The cells were fixed and permeablized and stained with a long red-labeled anti-BrdU antibody. S-phase cells were then quantitated by LSC. Fold increase in the percentage of cells in S-phase is graphed as shown in FIG. 14B.

TLR3/4 Specificity Upregulate Genes Involved in G1/S Transition.

TLR-mediated transcriptional upregulation was measured using Affymetrix Genechip microarray technology. Bone marrow-derived macrophages were treated for four hours with 100 nM CpG, 1 ng/ml Lipid A or 1 mg/ml poly I:C. Messenger RNA was harvested, labeled and used to probe the Mu11K Genechip set. Of the genes specifically activated by TLR3/4, a subset of genes involved in G1/S progression was identified. Some of these genes are presented in the dendogram in FIG. 15A. Upregulation is presented as red.

Cyclin D2 promotes the transition from G1 to S whereas Cycling G2 blocks the cell cycle at the G1/S transition (FIG. 15B). Activation of this combination would cause cells to accumulate at the G1/S transition.

### Example 4

The following Example describes that TLR3 activation decreases apoptosis in macrophage cell line.

The RAW 264.7 murine macrophage cell line was treated for two 24 h intervals with media alone (control), 10 ng/ml Lipid A (Lipid A) or 10 mg/ml poly I:C. In addition, as a positive control for apoptosis, another set of cells was treated with 3% H₂O₂ for 2 hours. Following fixation and permeablization, apoptosis was measured by the TUNNEL assay using the Death kit (Roche). Apoptotic cells are visualized by an increase in fluorescence (FITC) and detected by laser scanner cytometry (LSC). Lipid A treatment and poly I:C treatment were found to cause a 10 fold and 100 fold increase in apoptotic cells, respectively. The cell cycle is divided into G1 (red), S-phase (yellow) and G2/M (blue). Thus, TLR3/4 activation promotes apoptosis at all stages of the cell cycle (FIG. 16).

### Example 5

This Example illustrates that infection with live bacteria, Listeria monocytogenes (LM) activates the IRF3-IFNβ pathway and may influence development of adaptive immune responses.

Bone marrow-derived macrophages (BMMs) were infected with LM at a multiplicity of infection (MOI) of 1. At the indicated times, cells were harvested, fractionated for nuclear (top) and cytoplasmic (bottom) protein, and analyzed by immunoblotting using the indicated antibodies (FIG. 17A).

BMMs were infected with LM or E. coli at an MOI of 0.1 (left) or 10(right). At the indicated times cells were harvested for RNA, and gene expression was analyzed by Q-PCR using primers specific for IFNβ(top) or TNFα(bottom) (values relative to control L32 expression levels) (FIG. 17B).

Wildtype and interferon (alpha and beta) receptor (IFNAR)-deficient BMMs were infected as in (a) and RNA was harvested and analyzed by Q-PCR using primers specific for IL-15(top) or CD86 (bottom) (FIG. 17C).

BMMs were infected with LM for 4 hours after which antibiotic were added to the medium. At 24 post-infection, cells were analyzed for CD86 surface expression by flow cytometry (FIG. 17D). Quantification of data shown in FIG. 17D is shown in FIG. 17E.

Various publications are cited herein that are hereby incorporated by reference in their entirety.

As will be apparent to those skilled in the art to which the invention pertains, the present invention may be embodied in forms other than those specifically disclosed above without departing from the spirit or essential characteristics of the invention. The particular embodiments of the invention described above, are, therefore, to be considered as illustrative and not restrictive. The scope of the present invention is as set forth in the appended claims rather than being limited to the examples contained in the foregoing description.

What is claimed is:

1. A method for activating interferon regulatory factor 3 (IRF3) in a cell comprising contacting the cell with a molecule that stimulates a Toll-like receptors (TLR), thereby activating the IRF3 in the cell, wherein the cell expresses the TLR.

2. The method of claim 1, wherein the TLR is TLR3.

3. The method of claim 1, wherein the TLR is TLR4.

4. The method of claim 1, wherein the molecule that binds the TLR is a TLR ligand selected from a group consisting of bacterial antigen, LPS, lipid A, taxol, viral antigen, RSV F protein, double stranded RNA, imidazoquinoline compounds, and poly I:C.

5. A method for inhibiting a microbial infection comprising contacting the cell with a molecule that stimulates induction of IRF3 in the cell, thereby inhibiting the microbial infection.

6. The method of claim 5, wherein the inhibition of microbial infection is effected by inducing expression of primary response genes.

7. The method of claim 5, wherein the molecule that stimulates induction of IRF3 in the cell is a molecule that stimulates a TLR.

8. The method of claim 7, wherein the TLR is TLR3.

9. The method of claim 7, wherein the TLR is TLR4.

10. The method of claim 7, wherein the molecule that stimulates the TLR is a TLR ligand selected from a group consisting of a bacterial antigen, LPS, lipid A, taxol, a viral antigen, RSV F protein, double stranded RNA, imidazoquinoline compounds, and poly I:C.

11. The method of claim 6, wherein the primary response protein is any of IFIT1, ISG15, RANTES, IP10, or IFNβ.

12. The method of claim 6, wherein the microbial infection is a viral, fungal or bacterial infection.

13. A method for inhibiting a microbial infection by inducing activity of IFNβ in a cell comprising contacting the cell with a molecule that stimulates induction of IRF3.

14. The method of claim 13, wherein expression of IFNβ activates STAT1, thereby inducing activity of a secondary response protein in a cell.

15. The method of claim 14, wherein the secondary response protein is any one of Mx1, IFI1, IFI204, or IRF7.

16. The method of claim 13, wherein the microbial infection is a viral, fungal or bacterial infection.

17. The method of claim 13, wherein the molecule that stimulates activity of IRF3 is a TLR ligand selected from a group consisting of bacterial antigen, LPS, lipid A, taxol, viral antigen, RSV F protein, double stranded RNA, imidazoquinoline compounds, and poly I:C.

18. A method for inhibiting a microbial infection by stimulating induction of any one of MX1, IFI1, IFI204, IRF7, IFT3, IRG1, IRF9, IFI-TM31, PKR, EB13, or IFNα5 in a cell comprising contacting the cell with a molecule that stimulates induction of IFNβ, thereby stimulating induction of MX1, IFI1, IFI204, IRF7, IFT3, IRG1, IRF9, IFI1, IFI-TM31, PKR, EBI3, or IFNα5, so as to inhibit the microbial infection in the cell.

19. The method of claim 18, wherein the molecule that stimulates activity of IFNβ is a TLR ligand selected from a group consisting of bacterial antigen, LPS, lipid A, taxol, viral antigen, RSV F protein, double stranded RNA, imidazoquinoline compounds, and poly I:C.

20. A method for inhibiting viral replication in a cell by stimulating the TLR3/TLR4 and IRF3 pathways in the cell comprising contacting the cell with a molecule that stimulates the TLR3/TLR4 and IRF3 pathways, thereby inhibiting the viral replication in the cell.

21. The method of claim 20, wherein the molecule that stimulates the TLR3/TLR4 and IRF3 pathways is a TLR ligand selected from a group consisting of bacterial antigen, LPS, lipid A, taxol, viral antigen, RSV F protein, double stranded RNA, imidazoquinoline compounds, and poly I:C.

22. A method for inducing anti-inflammatory response in a cell by suppressing the TLR3/TLR4 and IRF3 pathways in the cell comprising contacting the cell with a molecule that suppresses the TLR3/TLR4 and IRF3 pathways, thereby inducing an anti-inflammatory response in the cell.

23. The method of claim 22, wherein the molecule that suppresses the TLR3/TLR4 and IRF3 pathways is a TLR ligand selected from a group consisting of a soluble TLR, an anti-TLR antibody, an anti-interferon antibody, an anti-LPS antibody, and molecules that block endotoxin shock.

24. A method for identifying small molecules that inhibit a microbial infection by activating any of the genes selected from a group consisting of MX1, IFI1, IFI204, and IRF7 in a cell, the method comprising contacting a cell with a molecule of interest that binds a TLR and activates the genes thereby inhibiting a microbial infection.


SEQUENCE LISTING (41 sequences)

SEQ ID NO: 1 - DNA, 23 bp, Artificial Sequence
  Description of Artificial Sequencequantitative real-time PCR (Q-PCR) amplification primer RANTES 5′
gcccacgtcaaggagtatttcta

SEQ ID NO: 2 - DNA, 20 bp, Artificial Sequence
  Description of Artificial Sequencequantitative real-time PCR (Q-PCR) amplification primer RANTES 3′
acacacttggcggttccttc

SEQ ID NO: 3 - DNA, 23 bp, Artificial Sequence
  Description of Artificial Sequencequantitative real-time PCR (Q-PCR) amplification primer Mx1 5′
aaacctgatccgacttcacttcc

SEQ ID NO: 4 - DNA, 24 bp, Artificial Sequence
  Description of Artificial Sequencequantitative real-time PCR (Q-PCR) amplification primer Mx1 3′
tgatcgtcttcaaggtttccttgt

SEQ ID NO: 5 - DNA, 21 bp, Artificial Sequence
  Description of Artificial Sequencequantitative real-time PCR (Q-PCR) amplification primer IFI1 5′
ccagagcatgggaaagaggtt

SEQ ID NO: 6 - DNA, 22 bp, Artificial Sequence
  Description of Artificial Sequencequantitative real-time PCR (Q-PCR) amplification primer IFI1 3′
ccggacctctgataggacactg

SEQ ID NO: 7 - DNA, 19 bp, Artificial Sequence
  Description of Artificial Sequencequantitative real-time PCR (Q-PCR) amplification primer IFI-204 5′
ttggctgcaatgggttcat

SEQ ID NO: 8 - DNA, 22 bp, Artificial Sequence
  Description of Artificial Sequencequantitative real-time PCR (Q-PCR) amplification primer IFI-204 3′
agtgggatattcattggttcgc

SEQ ID NO: 9 - DNA, 20 bp, Artificial Sequence
  Description of Artificial Sequencequantitative real-time PCR (Q-PCR) amplification primer IRF7 5′
acagggcgttttatcttgcg

SEQ ID NO: 10 - DNA, 19 bp, Artificial Sequence
  Description of Artificial Sequencequantitative real-time PCR (Q-PCR) amplification primer IRF7 3′
tccaagctcccggctaagt

SEQ ID NO: 11 - DNA, 19 bp, Artificial Sequence
  Description of Artificial Sequencequantitative real-time PCR (Q-PCR) amplification primer IP-10 5′
cctgcccacgtgttgagat

SEQ ID NO: 12 - DNA, 23 bp, Artificial Sequence
  Description of Artificial Sequencequantitative real-time PCR (Q-PCR) amplification primer IP-10 3′
tgatggtcttagattccggattc

SEQ ID NO: 13 - DNA, 21 bp, Artificial Sequence
  Description of Artificial Sequencequantitative real-time PCR (Q-PCR) amplification primer ISG-15 5′
caggacggtcttaccctttcc

SEQ ID NO: 14 - DNA, 21 bp, Artificial Sequence
  Description of Artificial Sequencequantitative real-time PCR (Q-PCR) amplification primer ISG-15 3′
aggctcgctgcagttctgtac

SEQ ID NO: 15 - DNA, 21 bp, Artificial Sequence
  Description of Artificial Sequencequantitative real-time PCR (Q-PCR) amplification primer IFIT1 5′
ggcaggaacaatgtgcaagaa

SEQ ID NO: 16 - DNA, 20 bp, Artificial Sequence
  Description of Artificial Sequencequantitative real-time PCR (Q-PCR) amplification primer IFIT1 3′
ctcaaatgtgggcctcagtt

SEQ ID NO: 17 - DNA, 21 bp, Artificial Sequence
  Description of Artificial Sequencequantitative real-time PCR (Q-PCR) amplification primer 18S 5′
ccgcggttctattttgttggt

SEQ ID NO: 18 - DNA, 19 bp, Artificial Sequence
  Description of Artificial Sequencequantitative real-time PCR (Q-PCR) amplification primer 18S 3′
ctctagcggcgcaatacga

SEQ ID NO: 19 - DNA, 22 bp, Artificial Sequence
  Description of Artificial Sequencequantitative real-time PCR (Q-PCR) amplification primer IFN-beta 5′
agctccaagaaaggacgaacat

SEQ ID NO: 20 - DNA, 22 bp, Artificial Sequence
  Description of Artificial Sequencequantitative real-time PCR (Q-PCR) amplification primer IFN-beta 3′
gccctgtaggtgaggttgatct

SEQ ID NO: 21 - DNA, 20 bp, Artificial Sequence
  Description of Artificial Sequencequantitative real-time PCR (Q-PCR) amplification primer IkappaBalpha 5′
ctgcaggccaccaactacaa

SEQ ID NO: 22 - DNA, 21 bp, Artificial Sequence
  Description of Artificial Sequencequantitative real-time PCR (Q-PCR) amplification primer IkappaBalpha 3′
cagcacccaaagtcaccaagt

SEQ ID NO: 23 - DNA, 26 bp, Artificial Sequence
  Description of Artificial Sequencequantitative real-time PCR (Q-PCR) amplification primer Beta Actin 5′
aggtgtgcaccttttattggtctcaa

SEQ ID NO: 24 - DNA, 22 bp, Artificial Sequence
  Description of Artificial Sequencequantitative real-time PCR (Q-PCR) amplification primer Beta Actin 3′
tgtatgaaggtttggtctccct

SEQ ID NO: 25 - DNA, 39 bp, Artificial Sequence
  Description of Artificial SequencePCR amplification primer IRF3(1-420)
caggactgatcaaccatggaaaccccgaaaccgcggatt

SEQ ID NO: 26 - DNA, 37 bp, Artificial Sequence
  Description of Artificial SequencePCR amplification primer IRF3-DBD(133-420)
caggacatccatgcactcccaggaaaacctaccgaag

SEQ ID NO: 27 - DNA, 37 bp, Artificial Sequence
  Description of Artificial SequencePCR amplification 3′ primer
caggacgcggccgcgatattccagtggcctggaagtc

SEQ ID NO: 28 - DNA, 23 bp, Artificial Sequence
  Description of Artificial SequencePCR primer IL-6 Forward
cacagaggataccactcccaaca

SEQ ID NO: 29 - DNA, 21 bp, Artificial Sequence
  Description of Artificial SequencePCR primer IL-6 Reverse
tccacgatttcccagagaaca

SEQ ID NO: 30 - DNA, 18 bp, Artificial Sequence
  Description of Artificial SequencePCR primer TLR3 Forward
tctggaaacgcgcaaacc

SEQ ID NO: 31 - DNA, 24 bp, Artificial Sequence
  Description of Artificial SequencePCR primer TLR3 Reverse
gccgttggactctaaattcaagat

SEQ ID NO: 32 - DNA, 21 bp, Artificial Sequence
  Description of Artificial SequencePCR primer TLR4 Forward
agaaattcctgcagtgggtca

SEQ ID NO: 33 - DNA, 24 bp, Artificial Sequence
  Description of Artificial SequencePCR primer TLR4 Reverse
tctctacaggtgttgcacatgtca

SEQ ID NO: 34 - DNA, 21 bp, Artificial Sequence
  Description of Artificial SequencePCR primer TIRAP Forward
caggcaggctctgttgaagaa

SEQ ID NO: 35 - DNA, 21 bp, Artificial Sequence
  Description of Artificial SequencePCR primer TIRAP Reverse
tgtgtggctgtctgtgaacca

SEQ ID NO: 36 - DNA, 22 bp, Artificial Sequence
  Description of Artificial SequencePCR primer MyD88 Forward
catggtggtggttgtttctgac

SEQ ID NO: 37 - DNA, 20 bp, Artificial Sequence
  Description of Artificial SequencePCR primer MyD88 Reverse
tggagacaggctgagtgcaa

SEQ ID NO: 38 - DNA, 20 bp, Artificial Sequence
  Description of Artificial SequencePCR primer ICAM1 Forward
tgtcagccactgccttggta

SEQ ID NO: 39 - DNA, 20 bp, Artificial Sequence
  Description of Artificial SequencePCR primer ICAM1 Reverse
caggatctggtccgctagct

SEQ ID NO: 40 - DNA, 19 bp, Artificial Sequence
  Description of Artificial SequencePCR primer L32 Forward
aagcgaaactggcggaaac

SEQ ID NO: 41 - DNA, 20 bp, Artificial Sequence
  Description of Artificial SequencePCR primer L32 Reverse
taaccgatgttgggcatcag